(19) United States
(12) Patent Application Publication
(10) Pub. No.: US 2004/0192676 A1
(43) Pub. Date: Sep. 30, 2004

(54) Ligands of melanocortin receptors and compositions and methods related thereto

(75) Inventors: Chen Chen, San Diego, CA (US); Fabio C. Tucci, San Diego, CA (US); Joe Anh Tran, San Marcos, CA (US); Caroline Chen, San Diego, CA (US); Nicole White, San Diego, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Neurocrine Biosciences, Inc., San Diego, CA

(21) Appl. No.: 10/742,592

(22) Filed: Dec. 19, 2003

Related U.S. Application Data
(60) Provisional application No. 60/435,922, filed on Dec. 20, 2002.

(51) Int. Cl. A61K 31/55; A61K 31/496; A61K 31/495; C 07D 4 9/02; C 07D 4 5/02

(52) U.S. Cl. 514/218; 514/252.13; 514/254.1; 514/255.01; 540/575; 544/374; 544/386

(57) ABSTRACT

Compounds which function as melanocortin receptor ligands and having utility in the treatment of melanocortin receptor-based disorders. The compounds have the following structure (I):

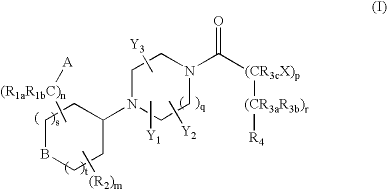

including stereoisomers, prodrugs, and pharmaceutically acceptable salts thereof, wherein A, B, m, n,p, q, r, s, t, R₁ₐ, R_{1b}, R₂, R₃ₐ, R_{3b}, R_{3c}, R₄, X, Y₁, Y₂, and Y₃ are as defined herein. Pharmaceutical compositions containing a compound of structure (I), as well as methods relating to the use thereof, are also disclosed.

### CROSS-REFERENCE TO RELATED APPLICATION

This application claims the benefit of U.S. Provisional Patent Application No. 60/435,922 filed Dec. 20, 2002, which application is incorporated herein by reference in its entirety.

### BACKGROUND OF THE INVENTION

1. Field of the Invention

This invention is generally directed to ligands of a melanocortin receptor, as well as to compositions and methods for using such ligands to alter activity of a melanocortin receptor.

2. Description of the Prior Art

Melanocortin (MC) receptors are members of the family of G-protein coupled receptors. To date, five distinct MC receptors (i.e., MC1-R, MC2-R, MC3-R, MC4-R and MC5-R) have been identified in a variety of tissues and these receptors have been shown to mediate a number of physiological processes. Ligands, including peptides and small molecules, have been shown to act as agonists or antagonists at these receptors.

The role of specific MC receptors in physiological processes has been the object of intense study since their discovery and cloning. These receptors are expressed in a variety of tissues including melanocytes, adrenal cortex, brain, gut, placenta, skeletal muscle, lung, spleen, thymus, bone marrow, pituitary, gonads and adipose tissue. A putative role of MC receptors has been shown in melanocytes, stimulatory actions on learning, attention and memory, motor effects, modification of sexual behavior, facilitation of nerve regeneration, anti-inflammatory and antipyretic effects, and the regulation of food intake and body weight.

The pro-opiomelanocortin (POMC) gene product is processed to produce a number of biologically active peptides that are expressed in the pituitary, and two locations in the brain: the arcuate nucleus of the hypothalamus and the solitary tract nucleus of the brain stem. These peptides elicit a range of biological activities. Two POMC peptides, α-melanocyte stimulating hormone (α-MSH) and adrenocorticotropic hormone (ACTH) control melanocyte and adrenocortical function, respectively, in the periphery.

Cloning studies have defined a family of five melanocortin (MC) receptors that respond to POMC peptides (reviewed in Rec. Prog. Hor. Res. 51:287-318, 1996). Each receptor in this family is pharmacologically distinct in its particular response to the POMC peptides α-MSH, γ-MSH and ACTH and to two peptide antagonists. Among the five receptors, MC4-R has the highest affinity for α-MSH. MC4-R differs from the other MC receptors in that it binds both natural melanocortin antagonists, agouti (Nature 371:799-802, 1994) and agouti-related protein (AgRP) (Biochem. Biophys. Res. Commun. 237:629-631, 1997). In contrast, MC1 -R only binds agouti, MC2-R does not bind AgRP, MC3-R only binds AgRP, and MC5-R has only low affinity binding for AgRP (Mol. Endocrinology 13:148-155, 1999).

The expression of specific MC receptors is restricted anatomically. MC1 -R is expressed primarily in melanocytes, while MC2-R is expressed in adrenocortical cells. MC3-R is expressed in brain, placenta and gut, and MC4-R is expressed primarily in the brain where its mRNA can be detected in nuclei that bind α-MSH. MC4-R is notably absent from adrenal cortex, melanocyte and placental tissues. Both MC3-R and MC4-R are expressed in arcuate and paraventricular neurons. MC5-R is expressed in brain, adipose tissues, muscle and exocrine glands.

α-Melanocyte stimulating hormone (α-MSH) is a tridecapeptide whose principal action (i.e., the activation of a set of G-protein coupled melanocortin receptors), results in a range of physiological responses including pigmentation, sebum production and feeding behavior. Cyclized peptide derivatives of α-MSH are potent modulators of these receptors. When administered by intracerebroventricular (i.c.v) injection into fasted animals, peptides exhibiting MCR-4 antagonist activity increase food intake and body weight. Moreover, overexpression of a naturally occurring peptide antagonist, agouti-related peptide (AgRP) has a similar effect on food intake and body weight. The development of small molecule antagonists of the MC4-R would selectively enhance the feeding response. MC4-R antagonists have a unique clinical potential because such compounds would stimulate appetite as well as decrease metabolic rate. Additionally, chronic MC4-R blockade causes an increase in lean body mass as well as fat mass, and the increase in lean body mass is independent of the increase in fat mass. Orally active forms of a small molecule MC4-R antagonist would provide a therapeutic strategy for indications in which cachexia is a symptom.

The MC receptors are also key mediators of steroid production in response to stress (MC2-R), regulation of weight homeostasis (MC4-R), and regulation of hair and skin pigmentation (MC1-R). They may have additional applications in controlling both insulin regulation (MC4-R) and regulation of exocrine gland fluction (MC5-R) (Cell 91:789-798, 1997); the latter having potential applications in the treatment of disorders such as acne, dry eye syndrome and blepharitis. Melanocortin peptides have also been reported to have anti-inflammatory activity, although the receptor(s) involved in mediating these effects have not yet been determined. Endocrine disorders such as Cushing's disease and congenital adrenal hyperplasia, which are characterized by elevated levels of ACTH, could be effectively treated with ACTH receptor (MC2-R) antagonists. Some evidence suggests that depression, which is characterized by elevated levels of glucocorticoids, may also be responsive to these same compounds. Similarly, elevated glucocorticoids can be an etiological factor in obesity. Synthetic melanocortin receptor agonists have been shown to initiate erections in men (J. Urol. 160:389-393, 1998). An appropriate MC receptor agonist could be an effective treatment for certain sexual disorders.

MC1 -R provides an ideal target for developing drugs that alter skin pigmentation. MC1-R expression is localized to melanocytes where it regulates eumelanin pigment synthesis. Two small clinical trials indicate that broad-spectrum melanocortin agonists induce pigmentation with limited side effects. The desired compound would have a short half-life and be topically applied. Applications include skin cancer prevention, UV-free tanning, inhibition of tanning and treatment of pigmentation disorders, such as tyrosinase-positive albinism.

The role of melanocortin receptors in regulation of adiposity signaling and food intake has been recently reviewed (Nature 404:661-669, 2000). Direct experimental evidence for the individual role of MC4 and MC3 receptors in energy homeostasis has not yet been reported due to the lack of potent and specific MC4 and MC3 agonists. Central administration of synthetic, non-selective MC-3R and MC4-R agonists, such as cyclic side-chain-lactam-modified peptide MT-II suppresses food intake in rodents and monkeys, and stimulates energy expenditure resulting in reduced adiposity (Endocrinology 142:2586-2592,2001). Conversely, selective peptide antagonists of the MC4 receptor stimulate food consumption and result in increased body weight, suggesting the main effects of agonist induced inhibition of food consumption are mediated by MC4-R receptor activity. (European J. Pharmacol. 405:25-32, 2000). Selective small molecule MC4-R antagonists also stimulate food intake in animal models of cachexia.

Genetically modified animals lacking the MC4-R receptor are hyperphagic and obese (Cell 88:131-141, 1997). Humans with defective melanocortin 4 receptors exhibit marked hyperphagia and increased body mass relative to their normal siblings (Nature Genet. 20:111-114, 1998). In addition, studies with mice lacking functional MC-3 receptors suggest that agonist stimulation of this receptor may also play a role in control of energy homeostasis, feeding efficiency, metabolism and bodyweight (Endocrinology 141:3518-3521, 2000). Therefore MC4-R and MC3-R agonists may be useful in the control of obesity and in treatment of related disorders including diabetes.

Due to their important biological role, a number of agonists and antagonists of the MC receptors have been suggested. For example, U.S. Pat. No. 6,054,556 is directed to a family of cyclic heptapeptides which act as antagonists for MC1, MC3, MC4 and MC5 receptors; U.S. Pat. No. 6,127,381 is directed to isoquinoline compounds which act upon MC receptors for controlling cytokine-regulated physiologic processes and pathologies; and published PCT Application No. WO 00/74679 is directed to substituted piperidine compounds that act as selective agonists of MC4-R. Published PCT Application No. WO01/05401 is directed to small peptides that are MC3-R specific agonists.

Accordingly, while significant advances have been made in this field, there is still a need in the art for ligands to the MC receptors and, more specifically, to agonists and/or antagonists to such receptors, particularly small molecules. There is also a need for pharmaceutical compositions containing the same, as well as methods relating to the use thereof to treat conditions associated with the MC receptors. The present invention fulfills these needs, and provides other related advantages.

### BRIEF SUMMARY OF THE INVENTION

In brief, this invention is directed to compounds that function as melanocortin (MC) receptor ligands. In this context, the term “ligand” means a molecule that binds or forms a complex with one or more of the MC receptors. This invention is also directed to compositions containing one or more compounds in combination with one or more pharmaceutically acceptable carriers, as well as to methods for treating conditions or disorders associated with MC receptors.

In one embodiment, this invention is directed to compounds that have the following structure (I):

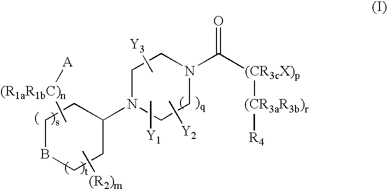

including stereoisomers, prodrugs, and pharmaceutically acceptable salts thereof, wherein A, B, m, n, p, q, r, s, t, R₁ₐ, R_{1b}, R₂, R₃ₐ, R_{3b}, R_{3c}, R₄, X, Y₁, Y₂ and Y₃ are as defined herein.

The compounds of this invention have utility over a broad range of therapeutic applications, and may be used to treat disorders or illnesses, including (but not limited to) eating disorders, obesity, inflammation, pain, chronic pain, skin disorders, skin and hair coloration, sexual dysfunction, dry eye, acne, anxiety, depression, and/or Cushing's disease. A representative method of treating such a disorder or illness includes administering an effective amount of a compound of this invention, preferably in the form of a pharmaceutical composition, to an animal (also referred to herein as a “patient”, including a human) in need thereof. The compound may be an antagonist or agonist or may stimulate a specific melanocortin receptor while functionally blocking a different melanocortin receptor.

Accordingly, in another embodiment, pharmaceutical compositions are disclosed containing one or more ligands of this invention in combination with a pharmaceutically acceptable carrier.

In one embodiment, the compounds of this invention are agonists to one or more MC receptors, and are useful in medical conditions where a melanocortin receptor agonist is beneficial. For example, the compounds of this invention may be utilized as MC4-R specific agonists or MC3-R specific agonists. Alternatively, the agonist may have mixed activity on the MC3 and MC4 receptor, and function as an antagonist of one of these receptors. In this context, the compounds of this invention may be used to treat obesity, erectile and/or sexual dysfunction, or diabetes mellitus.

In another embodiment, compounds of this invention may serve as antagonists to either the MC3-R or MC4-R receptor. Such antagonists have beneficial therapeutic effects, especially in the treatment of cachexia or wasting disease associated with cancer, AIDS, failure to thrive syndrome, and diseases associated with aging and senility. In more specific embodiments, the compounds are MC4-R antagonists for treatment of cachexia or wasting disease associated with cancer, AIDs, failure to thrive syndrome, and diseases associated with aging and senility.

These and other aspects of this invention will be apparent upon reference to the following detailed description and attached figures. To that end, certain patent and other documents are cited herein to more specifically set forth various aspects of this invention. Each of these documents is hereby incorporated by reference in its entirety.

### DETAILED DESCRIPTION OF THE INVENTION

As mentioned above, in one embodiment the present invention is generally directed to compounds having the following structure (I):

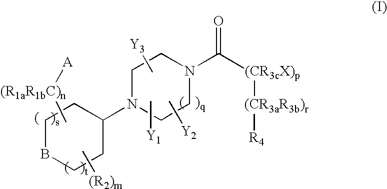

or a stereoisomer, prodrug or pharmaceutically acceptable salt thereof,

wherein:

A is —OR₅, —NR₆R₇, —C(═O)NR₆R₇, —C(═O)OR₈, —OC(═O)R₅, —OC(═O)NR₆R₇, —NR₆C(═O)OR₈, —NR₆C(═O)R₅, —NR₆C(═O)NR₆R₇, —NR₆SO₂R₉, —SO₂NR₆R₇, —NR₆SO₂NR₆R₇, —C(═NR₆)NR₆R₇, —C(═O)NR₆C(═NR₆)NR₆R₇, —NR₆C(═NR₇)R₉, heterocycle or substituted heterocycle;

B is a direct bond, —O—, —S—, —S(═O)—, or —S(═O)₂—;

m is 0, 1, or 2;

n is 0, 1, 2, or 3;

p is 0 or 1;

q is 1 or 2;

r is 0, 1, or 2;

s is 0, 1, or 2;

t is 0, 1, or 2;

X is, at each occurrence, independently hydrogen, hydroxy, fluorine, —OR₅, —NR₆R₇, —C(═O)NR₆R₇, —C(═O)OR₈, —OC(═O)R₅, —OC(═O)NR₆R₇, —NR₆C(═O)OR₈, —NR₆C(═O)R₅, —NR₆C(═O)NR₆R₇, —NR₆SO₂R₉, —SO₂NR₆R₇, —NR₆SO₂NR₆R₇, —C(═NR₆)NR₆R₇, —C(O)NR₆C(═NR₆)NR₆R₇, —NR₆C(═NR₇)R₉, heterocycle, or substituted heterocycle;

R₁ₐ and R_{1b} are, at each occurrence, the same or different and independently hydrogen, alkyl, substituted alkyl, aryl, substituted, aryl, hydroxy, amino, alkylamino, cyano, halide, —COOR₈, or —CONHR₆;

R₂ is, at each occurrence, independently alkyl, substituted alkyl, hydroxy, or halogen;

R₃ₐ, R_{3b}, and R_{3c} are, at each occurrence, the same or different and independently hydrogen, alkyl, or substituted alkyl;

R₄ is aryl, substituted aryl, heteroaryl, or substituted heteroaryl;

R₅ is, at each occurrence, independently hydrogen, hydroxy, alkyl, substituted alkyl, aryl, substituted aryl, heterocycle, or substituted heterocycle;

R₆ and R₇ are, at each occurrence, the same or different and independently hydrogen, alkyl, substituted alkyl, aryl, substituted aryl, arylalkyl, substituted arylalkyl, heterocycle, substituted heterocycle, heterocyclealkyl, or substituted heterocyclealkyl;

R₈ and R₉ are, at each occurrence, the same or different and independently hydrogen, alkyl, substituted alkyl, aryl, substituted aryl, arylalkyl, substituted arylalkyl, heterocycle, substituted heterocycle, heterocyclealkyl, or substituted heterocyclealkyl; and

Y₁, Y₂ and Y₃ are the same or different and independently hydrogen or alkyl, or Y₁ and Y₂ taken together are oxo

As used herein, the above terms have the following meaning:

“Alkyl” means a straight chain or branched, noncyclic or cyclic, unsaturated or saturated aliphatic hydrocarbon containing from 1 to 10 carbon atoms, while the term “lower alkyl” has the same meaning as alkyl but contains from 1 to 6 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, —CH₂cyclohexyl, and the like; while unsaturated cyclic alkyls include cyclopentenyl, cyclohexenyl, —CH₂cyclohexenyl, and the like. Cyclic alkyls are also referred to herein as a “homocycle”, and include bicyclic rings in which a homocycle is fused to a benzene ring. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“Alkanediyl” means a divalent alkyl from which two hydrogen atoms are taken from the same carbon atom or from different carbon atoms, such as —CH₂—, —CH₂CH₂—, —CH₂CH₂CH₂—, —CH(CH₃)CH₂—, -cyclopentane-, -cyclohexane-, -cycloheptane-, and the like.

“Aryl” means an aromatic carbocyclic moiety such as phenyl or naphthyl.

“Arylalkyl” means an alkyl having at least one alkyl hydrogen atom replaced with an aryl moiety, such as benzyl (i.e., —CH₂phenyl), —(CH₂)₂phenyl, —(CH₂)₃phenyl, —CH(phenyl)₂, and the like.

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, triazolyl, tetrazolyl, oxadiazolyl, benzoxadiazolyl, thiadiazolyl, indazolyl and quinazolinyl.

“Heteroarylalkyl” means an alkyl having at least one alkyl hydrogen atom replaced with a heteroaryl moiety, such as —CH₂pyridinyl, —CH₂pyrimidinyl, and the like.

“Heterocycle” (also referred to herein as a “heterocyclic ring”) means a 4- to 7-membered monocyclic, or 7- to 10-membered bicyclic, heterocyclic ring which is saturated, unsaturated, or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include heteroaryls as defined above. Thus, in addition to the heteroaryls listed above, heterocycles also include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Heterocyclealkyl” means an alkyl having at least one alkyl hydrogen atom replaced with a heterocycle, such as —CH₂morpholinyl, and the like.

“Oxo” means a divalent oxygen (i.e., ═O).

The term “substituted” as used herein means any of the above groups (i. e., alkyl, aryl, arylalkyl, heteroaryl, heteroarylalkyl, heterocycle and heterocyclealkyl) wherein at least one hydrogen atom is replaced with a substituent. In the case of an oxo substituent (“═O”) two hydrogen atoms are replaced. When substituted, “substituents” within the context of this invention include oxo, halogen, hydroxy, cyano, nitro, amino, alkylamino, dialkylamino, alkyl, alkoxy, thioalkyl, haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, —NRₐR_{b}, —NRₐC(═O)R_{b}, —NRₐC(═O)NRₐR_{b}, —NRₐC(═O)OR_{b} —NRₐSO₂R_{b}, C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ, —S(═O)₂ORₐ, —CH₂S(═O)₂Rₐ, —CH₂S(═O)₂NRₐR_{b}, ═NS(═O)₂Rₐ, and —S(═O)₂NRₐR_{b}, wherein Rₐ and R_{b} are the same or different and independently hydrogen, alkyl, substituted alkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, carbocycle, substituted carbocycle, carbocyclealkyl or substituted carbocyclealkyl.

“Halogen” means fluoro, chloro, bromo and iodo.

“Haloalkyl” means an alkyl having at least one hydrogen atom replaced with halogen, such as trifluoromethyl and the like.

“Alkoxy” means an alkyl moiety attached through an oxygen bridge (i.e., —O-alkyl) such as methoxy, ethoxy, and the like.

“Thioalkyl” means an alkyl moiety attached through a sulfur bridge (i.e., —S-alkyl) such as methylthio, ethylthio, and the like.

“Alkylamino” and “dialkylamino” mean one or two alkyl moiety attached through a nitrogen bridge (i.e., —N-alkyl) such as methylamino, ethylamino, dimethylamino, diethylamino, and the like.

“Mono- or di(cycloalkyl)methyl” represents a methyl group substituted with one or two cycloalkyl groups, such as cyclopropylmethyl, dicyclopropylmethyl, and the like.

“Alkylcarbonylalkyl” represents an alkyl substituted with a —C(═O)alkyl group.

“Alkylcarbonyloxyalkyl” represents an alkyl substituted with a —C(═O)Oalkyl group or a —OC(═O)alkyl group.

“Mono- or di(alkyl)amino represents an amino substituted with one alkyl or with two alkyls, respectively.

“Alkylamino” and “dialkylamino” mean one or two alkyl moiety attached through a nitrogen bridge (i.e., —N-alkyl) such as methylamino, ethylamino, dimethylamino, diethylamino, and the like.

In one embodiment, B is a direct bond, s is 1 and t is 2, and compounds of this invention have the following structure (Ia):

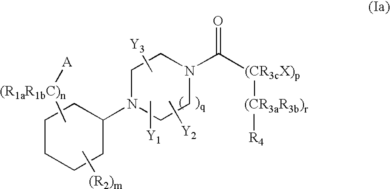

In another embodiment, B is O, s and t are both 1, and compounds of this invention have the following structure (Ib):

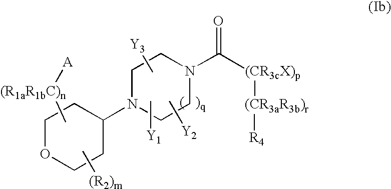

In a further embodiment, the “—(CR₁ₐR_{1b})ₙA” moiety is attached on the bridging carbon atom and compounds of this invention have the following structure (Ic):

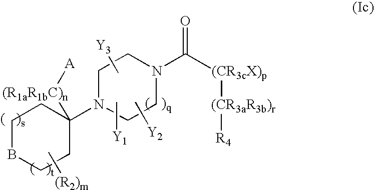

In still another embodiment, the “—(CR₁ₐR_{1b})ₙA” moiety is attached at a location other than the bridging carbon atom, a representative embodiment of which are compounds having the following structure (Id):

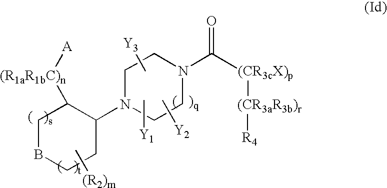

In yet a further embodiment, p is 1 and R_{3c} is hydrogen, and compounds of this invention have the following structure (Ie):

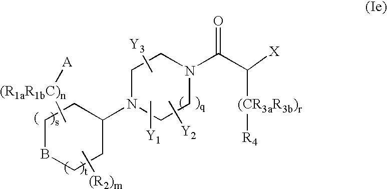

In addition, it should be understood that the various constituents as defined above in the context of structure (I), as well as the above substructures thereof, are not intended to be mutually exclusive. For example, “substituted alkyl” includes alkyls having at least one alkyl hydrogen atom replaced with a substituent (as that term is defined above), including substituents such as aryls (substituted or unsubstituted) and heterocycles (substituted or unsubstituted), and in the case of heterocyles further includes aromatic heterocycles—that is, heteroaryls (again, substituted or unsubstituted). Thus, an alkyl substituted with an aryl or a heterocycle moiety overlaps in scope with an arylalkyl or a heterocylcealkyl moiety, respectively. For example, methyl (an “alkyl” moiety) substituted with phenyl (an “aryl” moiety) is a benzyl moiety, which moiety is also encompasses within the scope of an “arylalkyl” moiety. Similarly, methyl (an “alkyl” moiety) substituted with pyridine (a “heterocycle” moiety) is a —CH₂pyridinyl moiety, which moiety is also encompassed within the scope of a “heterocyclealkyl” moiety and, more specifically, within the scope of a “heteroarylalkyl” moiety. In this regard, such aryl, heterocycle and/or heteroaryl moieties may be further substituted with on or more substituents as defined above.

The compounds of the present invention may be prepared by known organic synthesis techniques, including the methods described in more detail in the following Reaction Schemes and Examples. Piperazine-containing starting materials of this invention are commercially available, including those having a bridging heterocycle or subsituted heterocycle, are known in the literature and/or may be synthesized one skilled in this field. Furthermore, compounds of the present invention may be synthesized by a number of methods, both convergent and sequential, utilizing solution or solid phase chemistry.

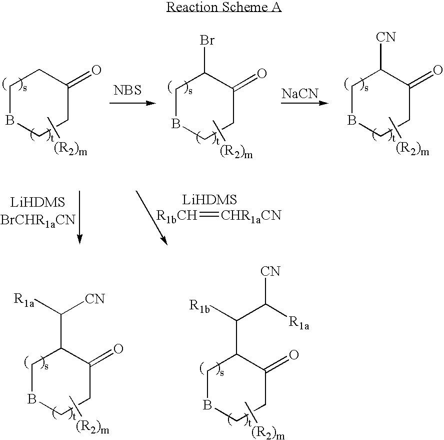

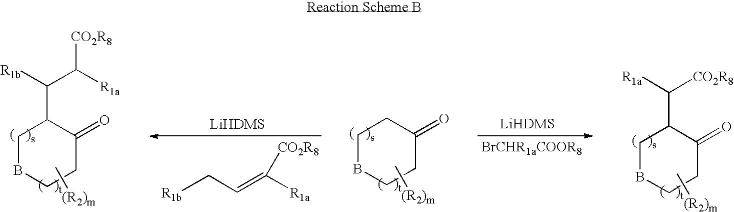

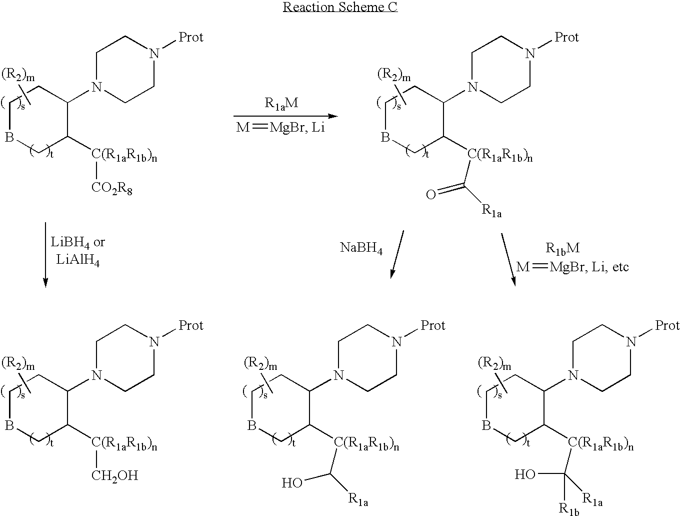

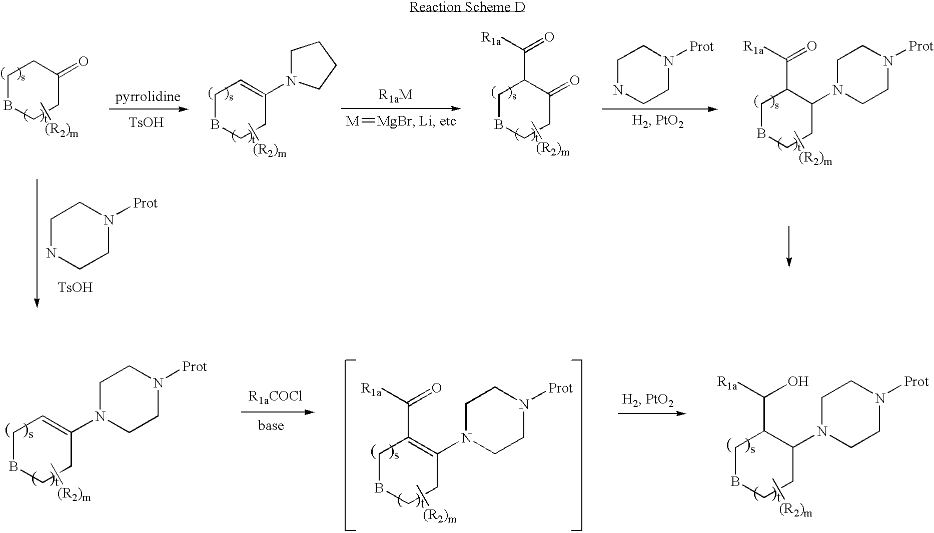

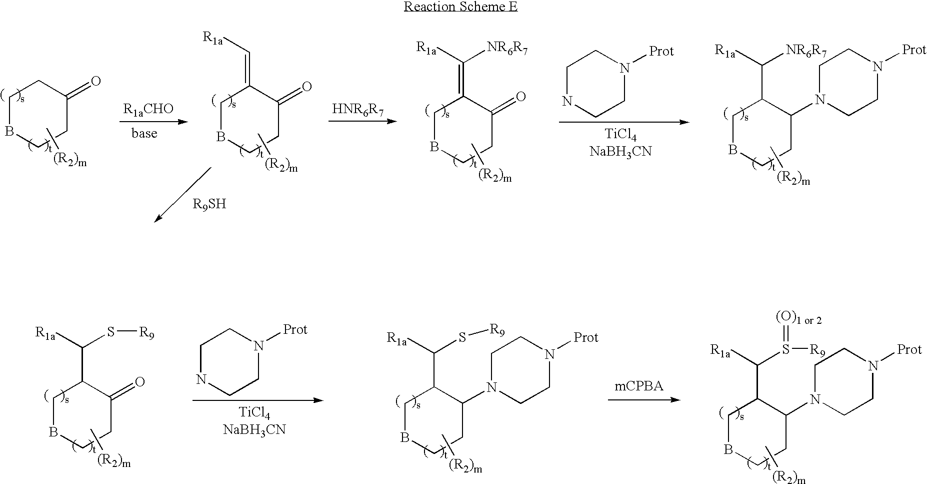

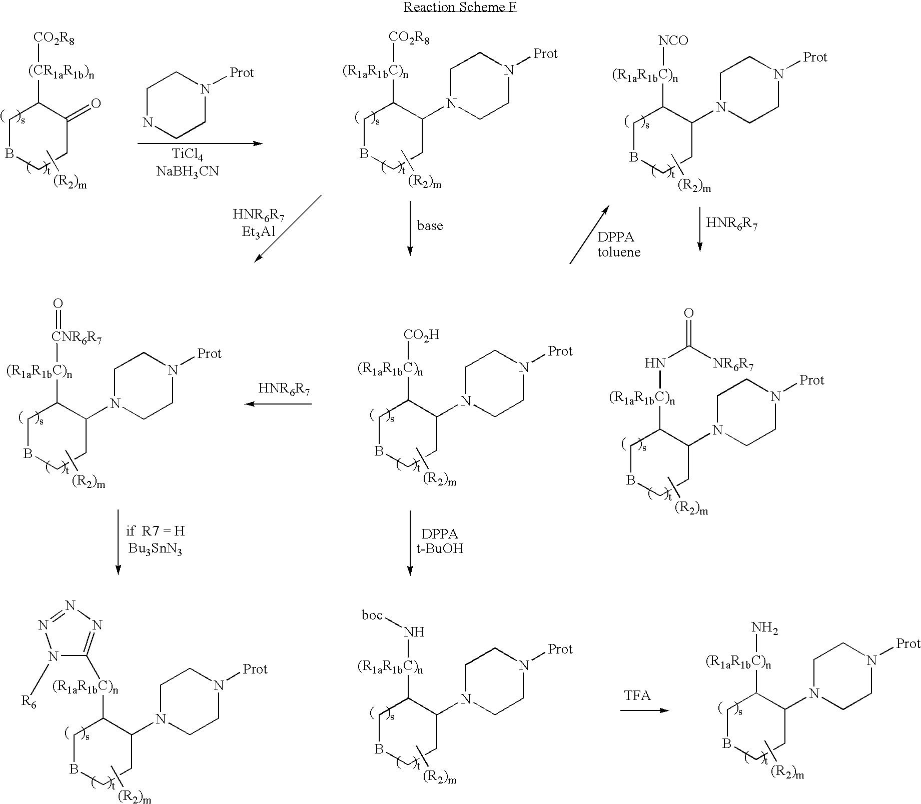

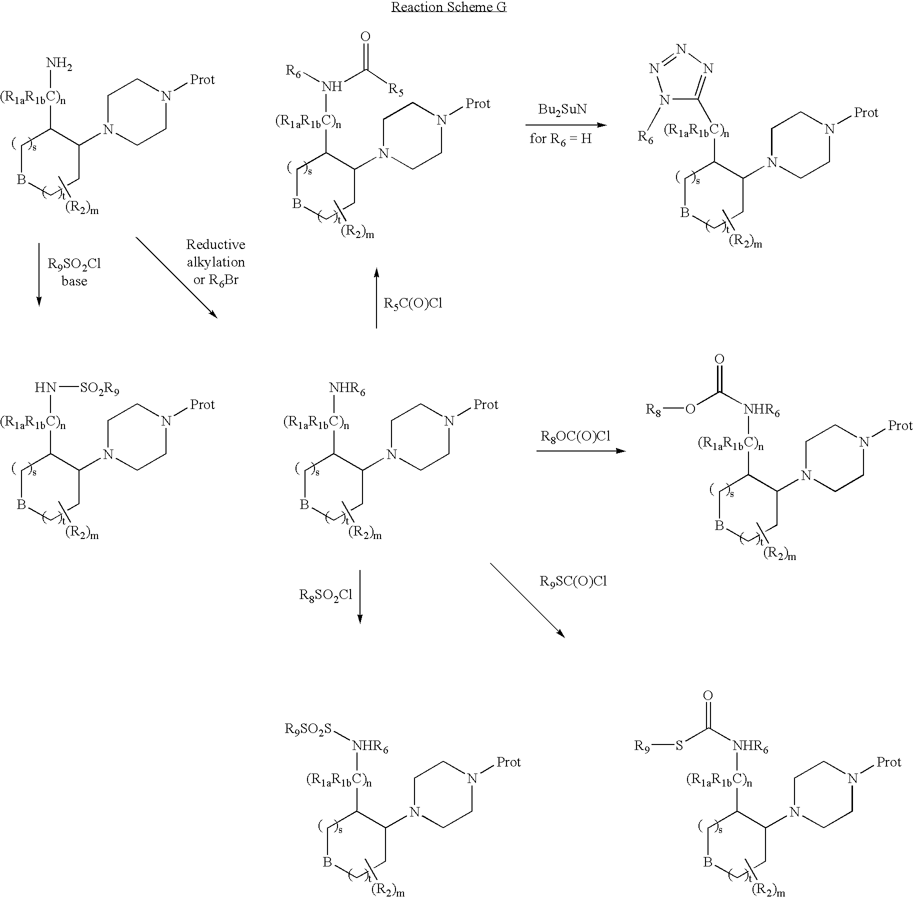

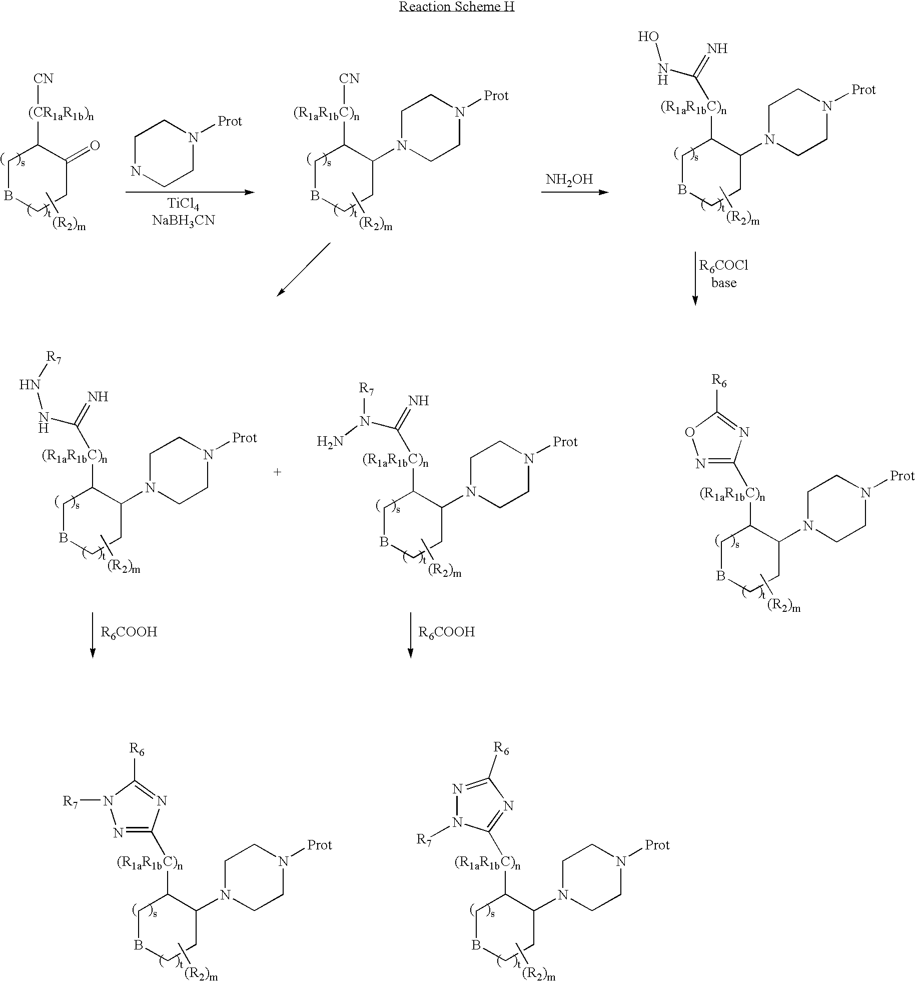

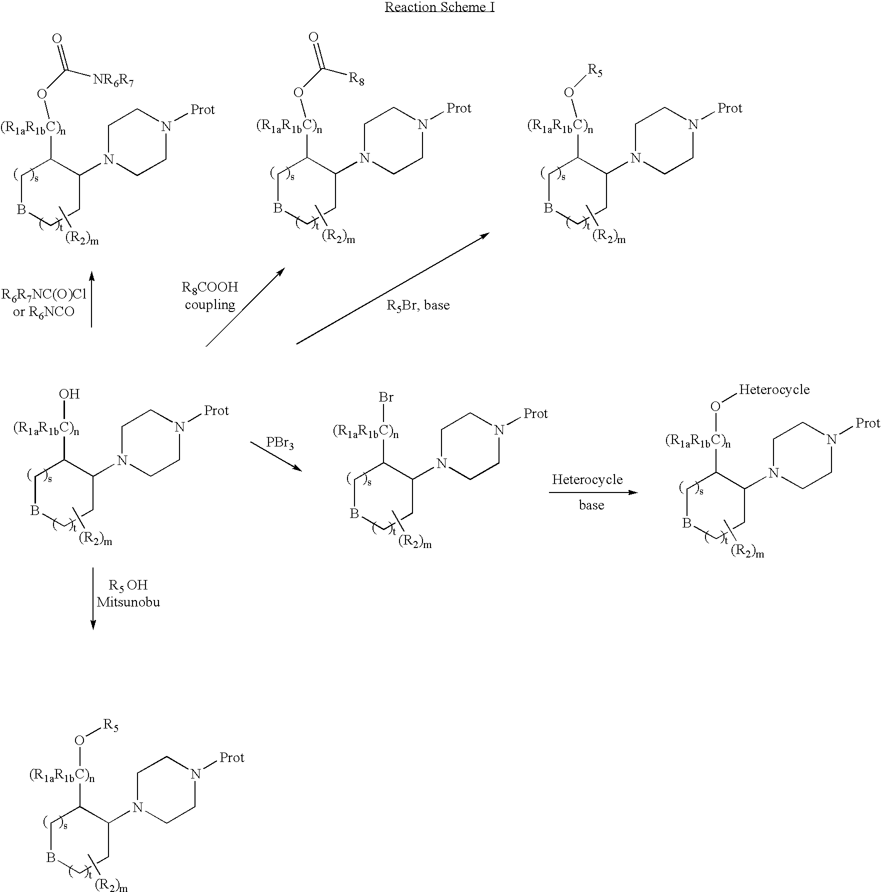

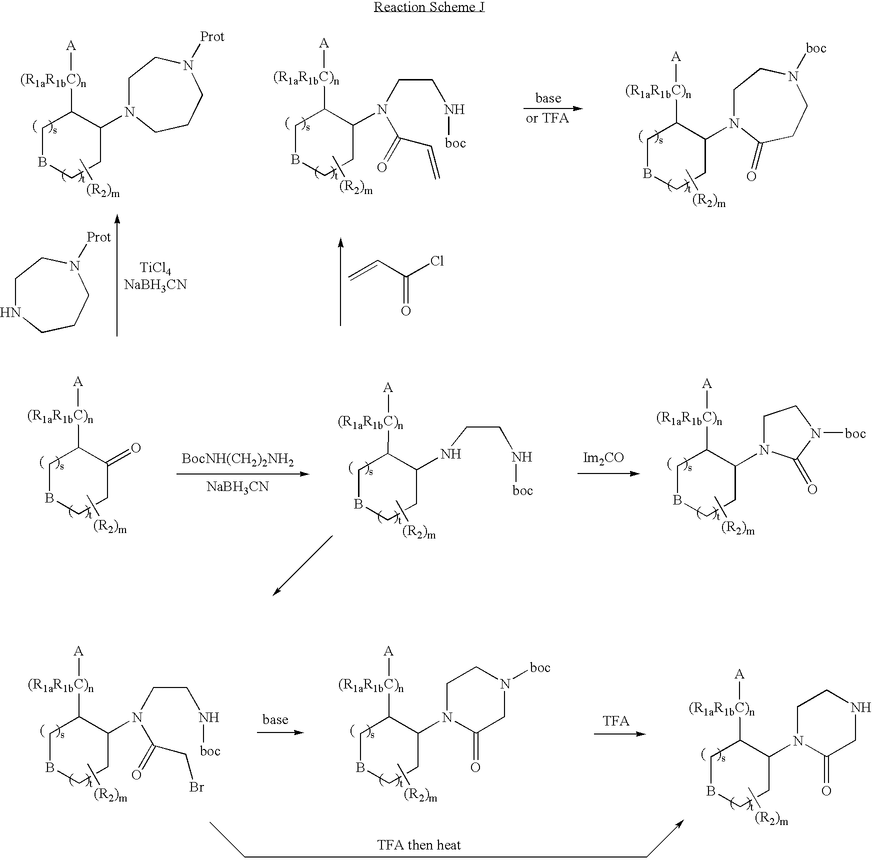

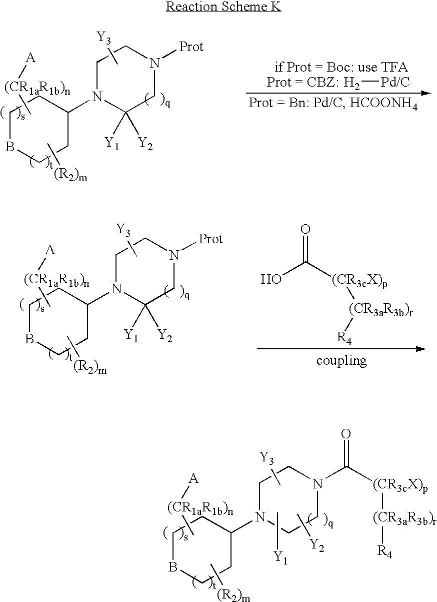

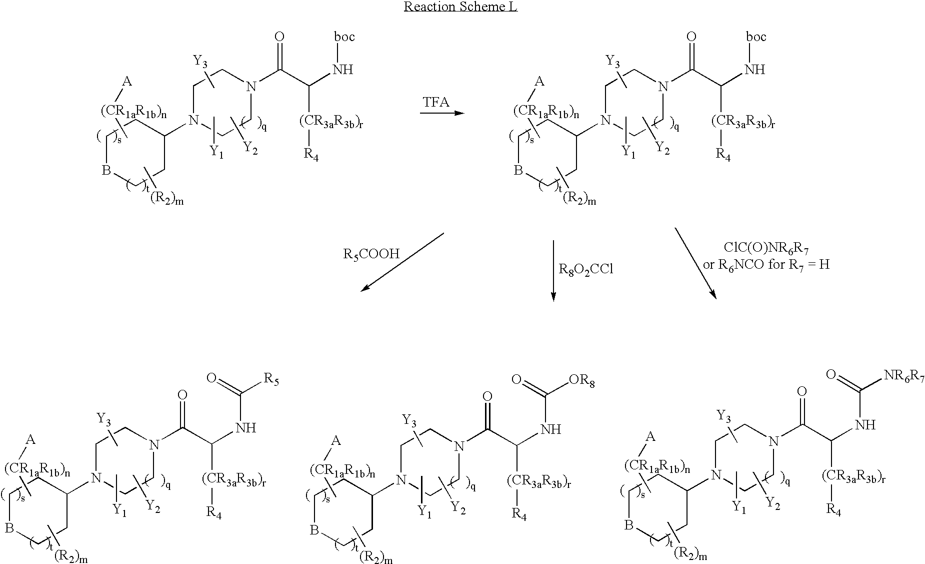

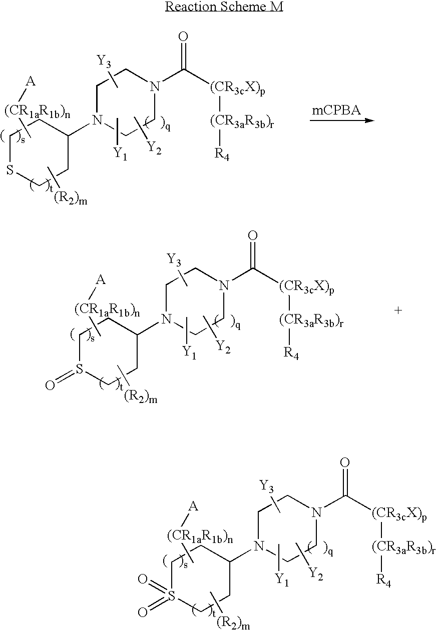

The compounds of the present invention may generally be utilized as the free acid or free base. Alternatively, the compounds of this invention may be used in the form of acid or base addition salts. Acid addition salts of the free amino compounds of the present invention may be prepared by methods well known in the art, and may be formed from organic and inorganic acids. Suitable organic acids include maleic, fumaric, benzoic, ascorbic, succinic, methanesulfonic, acetic, trifluoroacetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include hydrochloric, hydrobromic, sulfuric, phosphoric, and nitric acids. Base addition salts included those salts that form with the carboxylate anion and include salts formed with organic and inorganic cations such as those chosen from the alkali and alkaline earth metals (for example, lithium, sodium, potassium, magnesium, barium and calcium), as well as the ammonium ion and substituted derivatives thereof (for example, dibenzylammonium, benzylammonium, 2-hydroxyethylammonium, and the like). Thus, the term “pharmaceutically acceptable salt” of structure (I) is intended to encompass any and all acceptable salt forms.

In addition, prodrugs are also included within the context of this invention. Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol and amine functional groups of the compounds of structure (I). Further, in the case of a carboxylic acid (—COOH), esters may be employed, such as methyl esters, ethyl esters, and the like.

With regard to stereoisomers, the compounds of structure (I) may have chiral centers and may occur as racemates, racemic mixtures and as individual enantiomers or diastereomers. All such isomeric forms are included within the present invention, including mixtures thereof. Compounds of structure (I) may also possess axial chirality which may result in atropisomers. Furthermore, some of the crystalline forms of the compounds of structure (I) may exist as polymorphs, which are included in the present invention. In addition, some of the compounds of structure (I) may also form solvates with water or other organic solvents. Such solvates are similarly included within the scope of this invention.

The compounds of this invention may be evaluated for their ability to bind to a MC receptor by techniques known in this field. For example, a compound may be evaluated for MC receptor binding by monitoring the displacement of an iodonated peptide ligand, typically [¹²⁵I]-NDP-α-MSH, from cells expressing individual melanocortin receptor subtypes. To this end, cells expressing the desired melanocortin receptor are seeded in 96-well microtiter Primaria-coated plates at a density of 50,000 cells per well and allowed to adhere overnight with incubation at 37° C. in 5% CO₂. Stock solutions of test compounds are diluted serially in binding buffer (D-MEM, 1 mg/ml BSA) containing [¹²⁵I]-NDP-α-MSH (10⁵ cpm/ml). Cold NDP-α-MSH is included as a control. Cells are incubated with 50 μl of each test compound concentration for 1 hour at room temperature. Cells are gently washed twice with 250 μl of cold binding buffer and then lysed by addition of 50 μl of 0.5 M NaOH for 20 minutes at room temperature. Protein concentration is determined by Bradford assay and lysates are counted by liquid scintillation spectrometry. Each concentration of test compound is assessed in triplicate. IC₅₀ values are determined by data analysis using appropriate software, such as GraphPad Prizm, and data are plotted as counts of radiolabeled NDP-MSH bound (normalized to protein concentration) versus the log concentration of test compound.

In addition, functional assays of receptor activation have been defined for the MC receptors based on their coupling to Gₛ proteins. In response to POMC peptides, the MC receptors couple to G_{S} and activate adenylyl cyclase resulting in an increase in cAMP production. Melanocortin receptor activity can be measured in HEK293 cells expressing individual melanocortin receptors by direct measurement of cAMP levels or by a reporter gene whose activation is dependent on intracellular cAMP levels. For example, HEK293 cells expressing the desired MC receptor are seeded into 96-well microtiter Primaria-coated plates at a density of 50,000 cells per well and allowed to adhere overnight with incubation at 37° C. in 5% CO₂. Test compounds are diluted in assay buffer composed of D-MEM medium and 0.1 mM isobutylmethylxanthine and assessed for agonist and/or antagonist activity over a range of concentrations along with a control agonist α-MSH. At the time of assay, medium is removed from each well and replaced with test compounds or α-MSH for 30 minutes at 37° C. Cells are harvested by addition of an equal volume of 100% cold ethanol and scraped from the well surface. Cell lysates are centrifuged at 8000×g and the supernatant is recovered and dried under vacuum. The supernatants are evaluated for cAMP using an enzyme-linked immunoassay such as Biotrak, Amersham. EC₅₀ values are determined by data analysis using appropriate software such as GraphPad Prizm, and data are plotted as cAMP produced versus log concentration of compound.

As mentioned above, the compounds of this invention function as ligands to one or more MC receptors, and are thereby useful in the treatment of a variety of conditions or diseases associated therewith. In this manner, the ligands function by altering or regulating the activity of an MC receptor, thereby providing a treatment for a condition or disease associated with that receptor. In this regard, the compounds of this invention have utility over a broad range of therapeutic applications, and may be used to treat disorders or illnesses, including (but not limited to) eating disorders, cachexia, obesity, diabetes, metabolic disorders, inflammation, pain, skin disorders, skin and hair coloration, male and female sexual dysfunction, erectile dysfunction, dry eye, acne and/or Cushing's disease.

The compounds of the present invention may also be used in combination therapy with agents that modify sexual arousal, penile erections, or libido such as sildenafil, yohimbine, apomorphine or other agents. Combination therapy with agents that modify food intake, appetite or metabolism are also included within the scope of this invention. Such agents include, but are not limited to, other MC receptor ligands, ligands of the leptin, NPY, melanin concentrating hormone, serotonin or B₃ adrenergic receptors.

In another embodiment, pharmaceutical compositions containing one or more compounds of this invention are disclosed. For the purposes of administration, the compounds of the present invention may be formulated as pharmaceutical compositions. Pharmaceutical compositions of the present invention comprise a compound of structure (I) and a pharmaceutically acceptable carrier and/or diluent. The compound is present in the composition in an amount which is effective to treat a particular disorder of interest, and preferably with acceptable toxicity to the patient. Typically, the pharmaceutical composition may include a compound of this invention in an amount ranging from 0.1 mg to 250 mg per dosage depending upon the route of administration, and more typically from 1 mg to 60 mg. Appropriate concentrations and dosages can be readily determined by one skilled in the art.

Pharmaceutically acceptable carrier and/or diluents are familiar to those skilled in the art. For compositions formulated as liquid solutions, acceptable carriers and/or diluents include saline and sterile water, and may optionally include antioxidants, buffers, bacteriostats and other common additives. The compositions can also be formulated as pills, capsules, granules, or tablets that contain, in addition to a compound of this invention, dispersing and surface active agents, binders, and lubricants. One skilled in this art may further formulate the compound in an appropriate manner, and in accordance with accepted practices, such as those disclosed in Remington's Pharmaceutical Sciences, Gennaro, Ed., Mack Publishing Co., Easton, Pa. 1990.

In another embodiment, the present invention provides a method for treating a condition related to an MC receptor. Such methods include administration of a compound of the present invention to a warm-blooded animal in an amount sufficient to treat the condition. In this context, “treat” includes prophylactic administration. Such methods include systemic administration of compound of this invention, preferably in the form of a pharmaceutical composition as discussed above. As used herein, systemic administration includes oral and parenteral methods of administration. For oral administration, suitable pharmaceutical compositions include powders, granules, pills, tablets, and capsules as well as liquids, syrups, suspensions, and emulsions. These compositions may also include flavorants, preservatives, suspending, thickening and emulsifying agents, and other pharmaceutically acceptable additives. For parental administration, the compounds of the present invention can be prepared in aqueous injection solutions that may contain buffers, antioxidants, bacteriostats, and other additives commonly employed in such solutions.

The following examples are provided for purposes of illustration, not limitation.

### EXAMPLES

Aqueous Work Up

The reaction mixture was concentrated under a stream of nitrogen, taken up in dichloromethane, washed with aqueous sodium bicarbonate, and again concentrated. Final compounds were dissolved in methanol and filtered prior to preparative HPLC purification.

HPLC Columns and Gradients

Analytical HPLC columns were BHK laboratories ODS/0/13 30×75 mm, 5 μm, 120 A; the standard gradient was 1 mL/min 10-90% CH₃CN in water over 2 minutes, then 90% CH3CN for 1 minute. Constant percentage of 0.1% TFA was added.

Prep HPLC Column

YMC AQ, 5 μm, 120 A20, 20×50 mm cartridges

### Example 1

### Synthesis of Intermediate Compounds

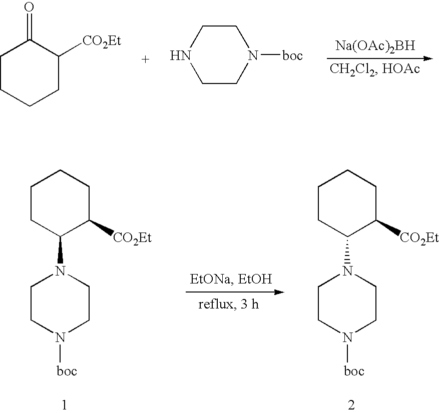

### Step 1A. cis-4-(2-Ethoxycarbonyl-cyclohexyl)-piperazine-1-carboxylic acid tert-butyl ester 1

A solution containing 2-oxo-cyclohexanecarboxylic acid ethyl ester (9.60 mL, 60.0 mmol), 1-Boc-piperazine (11.18 g, 60.0 mmol), HOAc (3.6 mL, 63.0 mmol) in dichloromethane (60 mL) was stirred at room temperature for 1.5 h. Sodium triacetoxy borohydride (31.79 g, 150.0 mmol) was added portionwise. The resulting white suspension was stirred vigorously at room temperature for 22 h. The reaction mixture was diluted with EtOAc (200 mL), and the organics were washed with H₂O, saturated NaHCO₃ and brine. After drying and concentration in vacuo, the resulting residue was chromatographed on silica-gel, eluting with a 4:1 v/v mixture of hexanes and EtOAc to give Compound 1 as a colorless oil. Yield: 5.45 g (16.0 mmol, 27%). LCMS m/z 341 (M⁺+1).

### Step 1 B. trans-4-(2-Ethoxycarbonyl-cyclohexyl)-piperazine-1-carboxylic acid tert-butyl ester 2

Sodium metal (460 mg, 20.0 mmol) was cut into small pieces and added portionwise to EtOH (50 mL), under N₂. When all solids dissolved, compound 1 (3.40 g, 10.0 mmol) was added and the resulting mixture was refluxed for 3 h. The reaction mixture was cooled, diluted with EtOAc (100 mL) and washed with H₂O. The organics were washed with brine, dried over anhydrous MgSO₄ and filtered. Concentration under vacuum gave a yellow oil that was purified by column chromatography (eluting with a 9:1 v/v mixture of hexanes and EtOAc) to give compound 2 as a thick yellow oil that solidified upon standing (1.60 g, 4.7 mmol, 47%). LCMS m/z 341 (M⁺+1).

### Example 2

### Synthesis of Representative Compounds

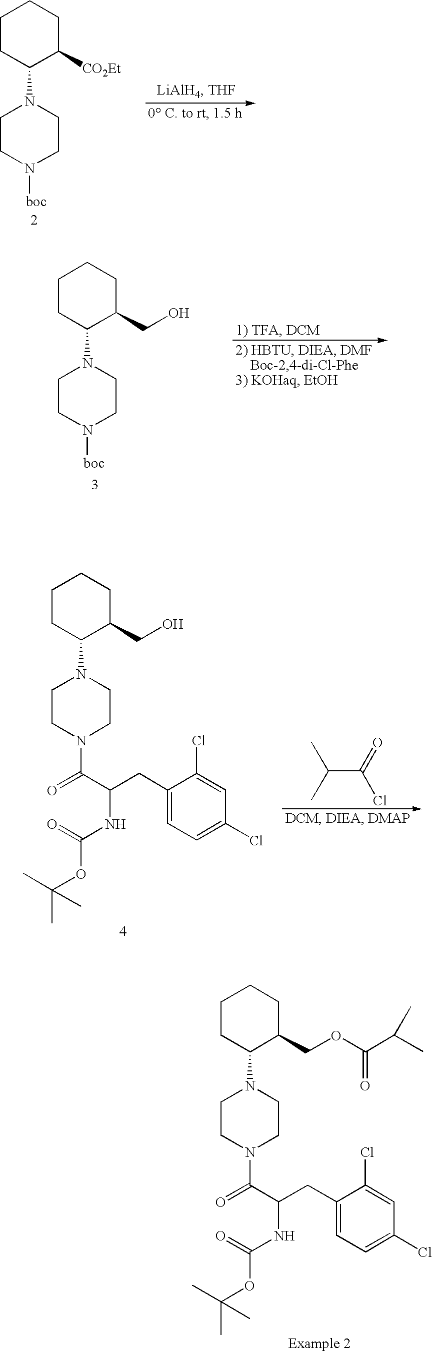

### Step 2A: trans-4-(2-Hydroxymethyl-cyclohexyl)-piperazine-1-carboxylic acid tert-butyl ester

trans-4-(2-Ethoxycarbonyl-cyclohexyl)-piperazine-1-carboxylic acid tert-butyl ester 2 (1.60 g, 4.7 mmol) was dissolved in THF (12 mL) and added slowly to a stirred suspension of LiAlH₄ (0.38 g, 9.4 mmol) in THF (23 mL), at 0° C. under N₂. The resulting mixture was stirred at 0° C. for 30 min. and then at room temperature for 30 min. The reaction mixture was cooled to 0° C., and quenched carefully by the addition of EtOAc (˜5 mL), followed by saturated Rochelle's salt solution (˜50 mL). EtOAc (100 mL) was added and the resulting white suspension was stirred vigorously for 30 min. The layers were separated, the organics were washed with brine, dried over anhydrous MgSO₄ and filtered. Evaporation gave the compound 3 as a white solid. Yield=1.40 g (4.7 mmol, 100%). LCMS m/z 299 (M⁺+1).

### Step 2B: {1-(2,4-Dichlorobenzyl)-2-[4-trans-(2-hydroxymethyl-cyclohexyl)-piperazin-1-yl]-2-oxo-ethyl}carbamic acid tert-butyl ester

trans-4-(2-Hydroxymethyl-cyclohexyl)-piperazine-1-carboxylic acid tert-butyl ester 3 (1.40 g, 4.7 mmol) was dissolved in dichloromethane (20 mL) and to that solution, trifluoroacetic acid (10 mL) was added. The resulting solution was stirred at room temperature for 7 h. The volatiles were removed in vacuo and the residue was then dissolved in DMF (10 mL) and treated with diisopropylethyl amine (1.80 mL, 10.3 mmol). This solution was set aside. In a separate flask, a solution containing (R)-Boc-2,4-dichlorophenylalanine (1.73 g, 5.2 mmol) and diisopropylethyl amine (1.80 mL, 10.3 mmol) in DMF (25 mL), was treated with O-benzotriazol-1-yl-N,N,N′, N′-tetramethyluronium hexafluorophosphate (HBTU, 2.32 g, 6.1 mmol). The resulting golden yellow solution was stirred at room temperature, under N₂, for 30 minutes. The solution previously set aside containing the deprotected piperazine was added, and the resulting mixture was stirred for 18 h at room temperature. The reaction was diluted with EtOAc (100 mL) and washed with 0.1 N HCl and then with saturated NaHCO₃. The organics were washed with brine, dried over anhydrous MgSO₄ and filtered. The residue was purified by column chromatography, eluting with a 3: 1, then a 2:1 v/v mixture of hexanes and EtOAc. The ester product was obtained as a light brown foam (1.83 g, 2.2 mmol, 94% yield based on (R)-Boc-2,4-dichlorophenylalanine). LCMS m/z 831 (M⁺+1). The above ester (1.75 g, 2.1 mmol) was dissolved in EtOH (5 mL) and treated with KOH (250 mg, 4.5 mmol) dissolved in H₂O (1 mL). The resulting mixture was refluxed for 2 h, cooled, diluted with H₂O (pH ˜8-9) and extracted with EtOAc. The organics were washed with brine, dried over anhydrous MgSO₄ and filtered. Evaporation gave the residue as an orange foam. Purification was performed by column chromatography on silica-gel, eluting with a 2:1 v/v mixture of EtOAc and hexanes, respectively. Compound 4 was isolated as a white foam (765 mg, 1.5 mmol, 71%). LCMS m/z 514 (M⁺+1).

### Step 2C

To a stirring solution containing {1-(2,4-dichloro-benzyl)-2-[4-trans-(2-hydroxymethyl-cyclohexyl)-piperazin-1-yl]-2-oxo-ethyl}-carbamic acid tert-butyl ester 4(40 mg, 77.8 μmol), diisopropylethyl amine (30 μL, 172.6 μmol) and DMAP (2 mg, 16.4 μmol) in dichloromethane (800 μL), was added isobutyryl chloride (26 mg, 240.0 μmol). The resulting mixture was stirred at room temperature at 16 h. The reaction mixture was evaporated and the residue was purified by preparative HPLC/MS yielding Example 2. (MH⁺=584)

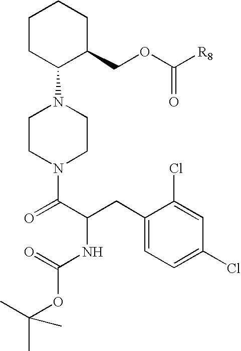

| Ex. # | R₈ | MS (MH+) | MW |
|---|---|---|---|
| 2-1 | isopropyl | 584 | 584.6 |
| 2-2 | methoxymethyl | 586 | 586.6 |
| 2-3 | cyclobutyl | 596 | 596.6 |
| 2-4 | phenyl | 618 | 618.6 |
| 2-5 | 3-methoxyphenyl | 648 | 648.6 |

### Example 3

### Synthesis of Representative Compounds

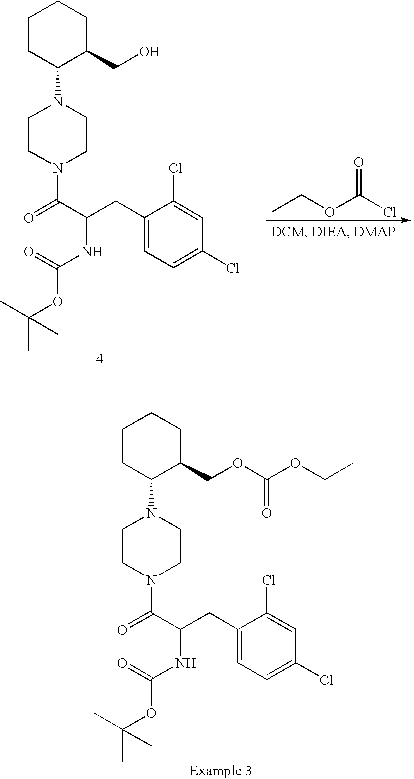

### Step 3A

To a stirring solution containing {1-(2,4-dichloro-benzyl)-2-[4-trans-(2-hydroxymethyl-cyclohexyl)-piperazin-1-yl]-2-oxo-ethyl}-carbamic acid tert-butyl ester 4 (40 mg, 77.8 μmol), diisopropylethyl amine (30 μL, 172.6 μmol) and DMAP (2 mg, 16.4 μmol) in dichloromethane (800 μL), was added ethylchloroformate (26 mg, 240.0 μmol). The resulting mixture was stirred at room temperature at 16 h. Example 3 was isolated by preparative HPLC/MS. (MH⁺=586).

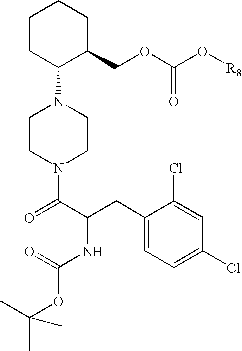

| Ex. # | R₈ | MS (MH+) | MW |
|---|---|---|---|
| 3-1 | ethyl | 586 | 586.6 |
| 3-2 | isobutyl | 614 | 614.6 |
| 3-3 | 2-fluoroethyl | 604 | 604.5 |
| 3-4 | phenyl | 634 | 634.6 |
| 3-5 | benzyl | 648 | 648.6 |

### Example 4

### Synthesis of Representative Compounds

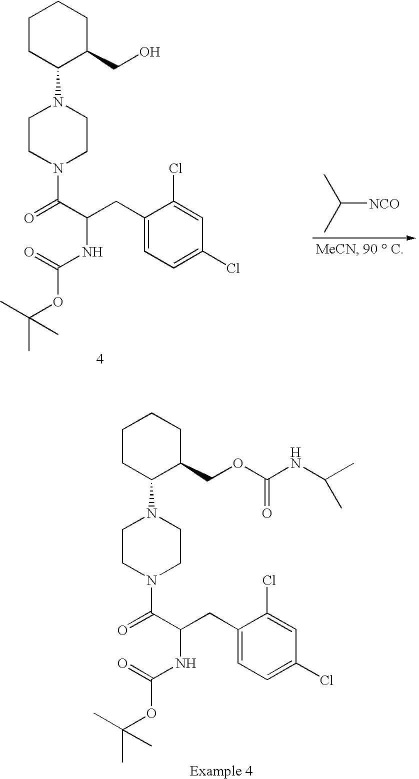

### Step 4A

To a stirring solution containing alcohol 75 (40 mg, 77.8 μmol) in acetonitrile (800 μL), the corresponding isocyanate was added (10 mg, 117.0 μmol). The mixture was heated to 90° C. for 8 h. The products were isolated after purification by preparative HPLC/MS.

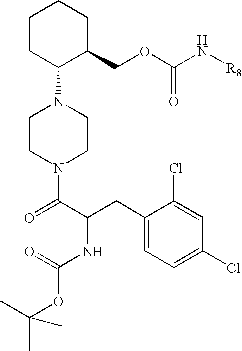

| Ex. # | R₈ | MS (MH+) | MW |
|---|---|---|---|
| 4-1 | isopropyl | 599 | 599.6 |
| 4-2 | ethyl | 585 | 585.6 |
| 4-3 | cyclohexyl | 639 | 639.7 |
| 4-4 | benzyl | 647 | 647.6 |

### Example 5

### Synthesis of Representative Compounds

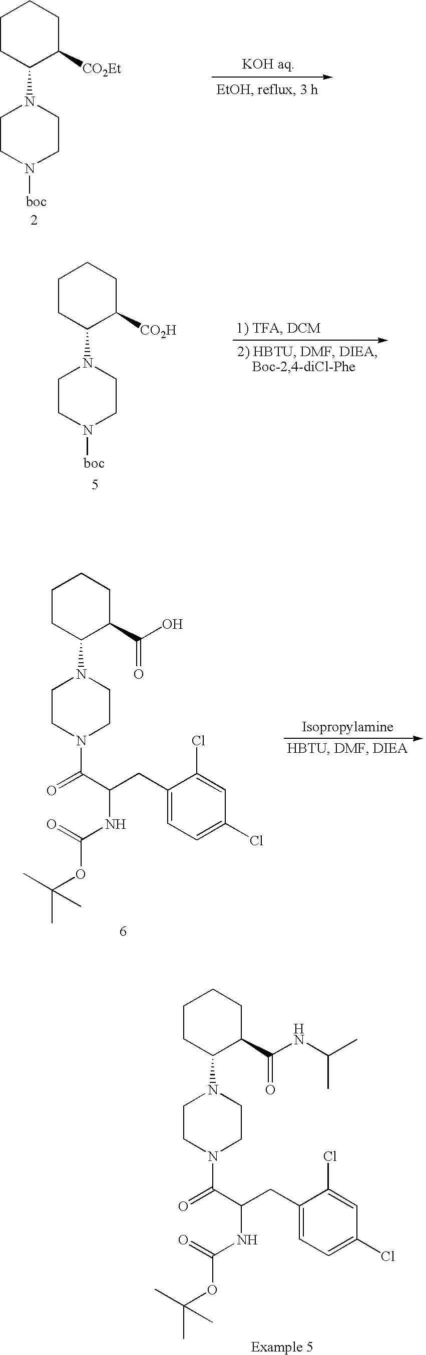

### Step 5A: 4-trans-(2-carboxy-cyclohexyl)-piperazine-1-carboxylic acid tert-butyl ester

A mixture containing trans-4-(2-ethoxycarbonyl-cyclohexyl)-piperazine-1-carboxylic acid tert-butyl ester 2 (800 mg, 2.4 mmol), KOH (260 mg, 4.6 mmol), EtOH (5 mL) and H₂O (1 mL) was refluxed for 3 hours. The reaction mixture was cooled, diluted with H₂O and acidified to pH ˜1 with a 1 N HCl solution. After ethyl acetate extraction, the organics were separated, washed with brine, dried over anhydrous MgSO₄, filtered and evaporated. Compound 5 was obtained as a tan oil and was used in the next step without further purification. Yield=173 mg (0.6 mmol, 28%). LCMS m/z 313 (M⁺+1).

### Step 5B: trans-2-{4-[2-tert-butoxycarbonylamino-3-(2,4-dichloro-phenyl)-propionyl]-piperazin-1-yl}-cyclohexanecarboxylic acid

4-trans-(2-carboxy-cyclohexyl)-piperazine-1-carboxylic acid tert-butyl ester 5 (173 mg, 0.6 mmol) was dissolved in dichloromethane (6 mL) and to that solution, trifluoroacetic acid (3 mL) was added. The resulting solution was stirred at room temperature for 3 hours and the volatiles were removed in vacuo. The deprotected piperazine was then dissolved in DMF (2 mL) and treated with diisopropylethyl amine (700 μL, 4.0 mmol). This solution was set aside. In a separate flask, a solution containing (R)-Boc-2,4-dichlorophenylalanine (267 g, 0.8 mmol), diisopropylethyl amine (350 μL, 2.0 mmol) in DMF (4 mL), was treated with HBTU (417 mg, 1.1 mmol). The resulting golden yellow solution was stirred at room temperature, under N₂, for 30 minutes. The solution containing the deprotected piperazine was added, and the resulting mixture was stirred for 18 h at room temperature. The reaction was diluted with EtOAc (20 mL) and washed with 0.1 N HCl and then with saturated NaHCO₃. The organics were washed with brine, dried over anhydrous MgSO₄ and filtered. The residue was purified by preparative HPLC/MS. Compound 6 was obtained as a white solid (25 mg, 47.4 μmol, 8%). LCMS m/z 528 (M⁺+1).

### Step 5C: {1-(2,4-dichloro-benzyl)-trans-2-[4-(2-isopropylcarbamoyl-cyclohexyl)-piperazin-1-yl]-2-oxo-ethyl}-carbamic acid tert-butyl ester

trans-2-{4-[2-tert-butoxycarbonylamino-3-(2,4-dichloro-phenyl)-propionyl]-piperazin-1-yl}-cyclohexanecarboxylic acid 6 (19 mg, 36.1 μmol) was dissolved in DMF (1 mL) and treated with diisopropylethyl amine (15 μL, 86.3 μmol). To this mixture, HBTU (18 mg, 47.5 mmol) was added. The resulting solution was stirred at room temperature for 35 minutes and treated with isopropylamine (15 μL, 175.1 μL). After 2 hours at room temperature, the volatiles were removed in vacuo and the residue purified by preparative HPLC/MS to give Example 5. LCMS m/z 569 (M⁺+1).

### Example 6

### Synthesis of Representative Compounds

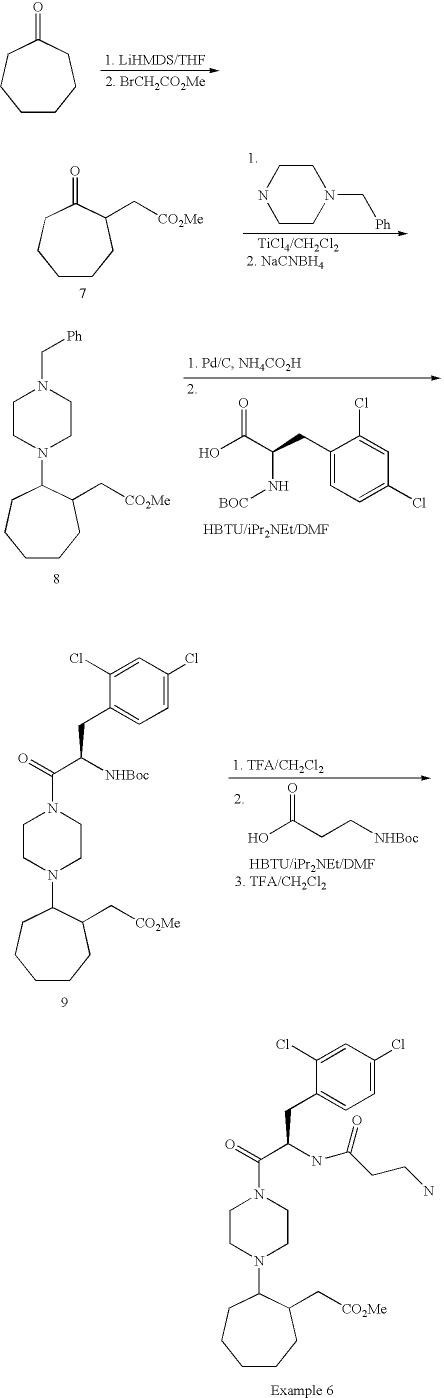

### Step A. 2-(methoxycarbonylmethyl)cycloheptanone

To a solution of cycloheptanone (5.53 mL, 46.9 mmol) in THF (200 mL) at −78° C. was added LiHMDS (1.0 M in pentane, 51.6 mL, 51.6 mmol). After 0.5 h, methyl bromoacetate (4.9 mL, 51.6 mmol) was added to the reaction dropwise. The reaction was stirred at the same temperature for 2 h and then was quenched with saturated aqueous ammonium chloride solution (200 mL). The mixture was warmed to room temperature then extracted with diethyl ether. The combined organic layers were washed with brine, dried (Na₂SO₄), filtered and evaporated. Silica-gel chromatography (2:1 hexanes: ethyl acetate) afforded the ketone 7 (5.00 g, 27.1 mmol) in 58% yield. ¹H NMR (300 MHz, CDCl₃) δ3.66 (s, 3H), 3.17-3.06 (m, 1H), 2.83 (dd, J=16.8, 8.4 Hz, 1H), 2.65 (dtd, J=16.2, 4.7, 1.5 Hz, 1H), 2.45 (J=15.6, 10.8, 4.8 Hz, 1H), 2.30 (dd, J=16.8, 5.7 Hz, 1H), 1.99-1.20 (m, 8H). GCMS, 184 (M+1).

### Step B. 2-(methoxycarbonylmethyl)-1-(4-benazlpiperazine)cycloheptane

A solution of 2-(methoxycarbonylmethyl)cycloheptanone 7 (2.5g, 13.6 mmol) and 1-benzylpiperazine in methylene chloride (70 mL) was cooled to 0° C. and a solution of titanium (IV) chloride (27 mL, 27 mmol) was added dropwise. The mixture was warmed to room temperature and stirred for a further 18 h. Sodium cyanoborohydride (4.0g, 63.5 mmol) was added in portions and the mixture stirred for a further 5 h after which time, it was poured on to saturated aqueous sodium hydrogen carbonate solution. The mixture was extracted twice with methylene chloride and the organic layer was washed with brine, dried (MgSO₄), filtered and evaporated. Silica-gel chromatography of the residue (2:1 hexanes:ethyl acetate) afforded compound 8 as the major isomer (0.88g, 19%) as an oil and the minor isomer (0.18 g, 4%) as an oil. ¹H NMR (major isomer) (300 MHz, CDCl₃) δ7.32-7.20 (m, 5H,), 3.60 (s, 3H), 3.52 (s, 2H), 2.70-2.64 (m, 4 H), 2.50-2.28 (m, 8H), 2.15-2.04 (m, 4 H), 1.74-1.20 (m, 10H). LCMS 345, M+1

### Step C. 2-(methoxycarbonylmethyl)-1-piperazinecycloheptane

A solution of the benzyl protected 2-(methoxycarbonylmethyl)-1-(4-benzylpiperazine)cycloheptane 8 (0.86g, 2.5 mmol) in methanol (15 mL) was de-gassed with nitrogen then 10% Pd on carbon (0.4 g) followed by ammonium formate (0.48 g, 7.5 mmol) was added. The mixture was refluxed for 2 h after which time, TLC showed the disappearance of starting material. The mixture was filtered and the solvent was evaporated. The residue was taken up in methylene chloride and again filtered and evaporated to afford the deprotected piperazine (0.63 g, 2.5 mmol). This material was used without further purification. ¹H NMR (300 MHz, CDCl₃) δ8.43 (s, 1H) 3.67 (s, 3 H), 3.08-3.00 (m, 4 H), 2.91-2.86 (m, 2H), 2.61-2.51 (m, 2 H), 2.20-2.06 (m, 4 H), 1.74-1.31 (m, 10H). LCMS (255, M+1)

### Step D. 1-[2-(methoxycarbonylmethyl)cyclopentyl]-4-[(2R)-(tert-butoxycarbonylamino)-3-(2,4-dichlorophenyl)propionyl]piperazine

A mixture of HBTU (0.98 g, 2.6 mmol) and (R)-N-tert-butoxycarbonyl 2,4-dichlorophenylalanine (0.87 g, 2.6 mmol) in dry DMF (5 mL) was stirred at room temperature and diisopropylethylamine (0.90 mL, 5.2 mmol) was added dropwise. After stirring for 30 mins, the mixture was added to the 2-(methoxycarbonylmethyl)-1-piperazinecycloheptane (from step 6C, 0.63 g, 2.5 mmol). The mixture was stirred for 16 h after which time water was added, and the mixture twice extracted with methylene chloride. The organic layer was washed with water (3×), brine, dried (MgSO₄) and evaporated. Silica-gel chromatography of the residue afforded the amide 9 (0.57g, 40%) as an oil). ¹H NMR (300 MHz, CDCl₃) d 7.38 (s, 1 H), 7.15 (s, 2 H), 5.46-5.43 (m, 1H), 4.99-4.92 (m, 1 H), 3.63 (s, 2H), 3.60-3.54 (m, 1H), 3.40-3.26 (m, 3H), 3.01-2.98 (m, 2 H), 2.68-2.52 (m, 2H), 2.14-2.04 (m, 4H), 1.58-1.25 (m, 20H).

### Step E. 1-[2-(methoxycarbonylmethyl)cyclopentyl]-4-[(2R)-(3-aminopropionylammido)-3-(2,4-dichlorophenyl)propionyl]piperazine

A solution of 1-[2-(methoxycarbonylmethyl)cyclopentyl]-4-[(2R)-(tert-butoxycarbonylamino)-3-(2,4-dichlorophenyl)propionyl]piperazine 9 (0.035 g, 0.062 mmol) in methylene chloride (1 mL) was treated with TFA (0.5 mL) and stirred at room temperature until LCMS showed disappearance of the starting material and presence of the free amine (LCMS, 470 M+1). The solvent was evaporated and the residue was treated with saturated aqueous sodium hydrogen carbonate solution. The mixture was extracted with methylene chloride and the organic extracts were dried (MgSO₄), filtered and evaporated to afford the free amine. In a separate flask, diisopropylethylamine (0.024 mL, 0.136 mmol) was added to a mixture of N-tert-butoxycarbonyl B-alanine (0.024 g, 0.124 mmol) and HBTU (0.047g, 0.124 mmol) in dry DMF (0.5 mL). After stirring for 30 min., the mixture was added to the free amine and the resulting mixture stirred for 18 hr. Water was added and the mixture was extracted with methylene chloride and the organic layers were dried (MgSO₄), filtered and evaporated. The residue was dissolved in methylene chloride and treated with TFA and stirred until LCMS indicated removal of the boc-group. After evaporation of the solvent, the residue was purified by HPLC to afford Example 6. LCMS 542.

The following compounds were prepared with a similar procedure.

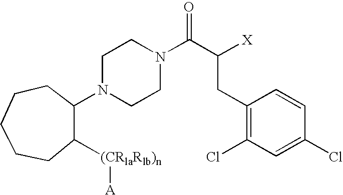

| Ex. # | —(CR₁ₐR_{1b})—A | —X | MS (MH⁺) |
|---|---|---|---|
| 6-1 | —COOMe | —NH₂ | 471 |
| 6-2 | —COOMe | —NHCOCH₂CH₂NH₂ | 542 |
| 6-3 | —COOMe | —NHCOCH₂NH₂ | 528 |
| 6-4 | —COOMe | —NHCOCH₂NMe₂ | 556 |
| 6-5 | —COOMe | —NHCOMe | 513 |
| 6-6 | —COOMe | —NHCOOEt | 543 |
| 6-7 | —COOMe | —NHCONHEt | 542 |
| 6-8 | —1-triazole | —NHCOMe | 522 |
| 6-9 | —1-triazole | —NHCOOEt | 552 |
| 6-10 | —1-triazole | —NHCONHEt | 551 |

### Example 7

### 1-[2-Methyl-3-(4-Chlorophenyl)Propionyl-4-[1-(Phenylacetamido)Methyl]Cyclohexylpiperazine

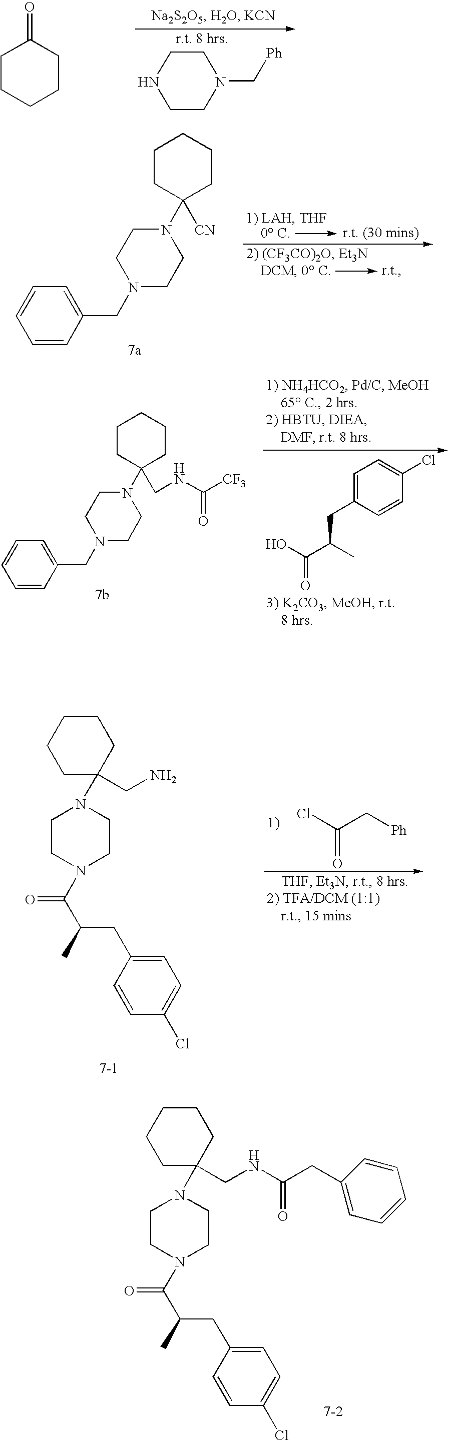

### Step 7A: 1-(1-Cyanocyclohexyl)-4-benzylpiperazine 7a

Cyclohexanone (7.3 mL, 70 mmol) was dissolved in water (140 mL) along with Na₂S₂O₅ (6.4 g, 35 mmol). The mixture was allowed to stir at room temperature for 1.5 hours then 1-benzylpiperazine (12.2 mL, 70 mmol) was added. The mixture was stirred for 2 hours and KCN (4.8 g, 74 mmol) was added to the reaction mix. The reaction mixture was then allowed to stir at room temperature overnight. The product was then extracted with dichloromethane (3×200 mL). The combined extracts were dried over anhydrous MgSO₄, filtered, and solvent was removed under vacuum. Compound 7a was obtained as a white solid in quantitative yield.

### Step 7B: 1-[1-(Trifluoroacetamidomethyl)cyclohexyl]-4-benzylpiperazine 7b

1-(1-Cyanocyclohexyl)-4-benzylpiperazine 7a (10 g, 35.3 mmol) was dissolved in ether (176 mL) and added dropwise to a mixture of LiAlH₄ (2.7 g, 71 mmol) in ether (353 mL) at room temperature. After the addition, the mixture was allowed to stir at room temperature for 0.5 hours. The reaction was then quenched by adding 2 mL of H₂O, followed by 1.5 mL of 20% NaOH, then 7 mL of H₂O. The reaction mixture was then filtered through celite and the residue was washed with ether. The ethereal mother liquor was dried over anhydrous MgSO₄ and solvent was removed under vacuum. The intermediate amine product was recovered in 94% yield without any further purification. This amine intermediate (9.5 g, 33 mmol) was then dissolved in dichloromethane (100 mL) along with Et₃N (4.8 mL, 34.7 mmol) and the reaction mixture was cooled to 0° C. To the reaction flask, trifluoroacetic anhydride (4.9 mL, 34.7 mmol) was added and the reaction was stirred at 0 ° C for 10 minutes then at room temperature for 4 hours. Compound 7b was obtained as a clear oil (quantitative yield) after the reaction mixture was concentrated under vacuum. No further purification was needed.

### Step 7C: 1-[1-(Trifluoroacetamidomethyl)cyclohexyl]-4-[2R-methyl-3-(4-chlorophenyl)propionyl]piperazine

1-[1-(Trifluoroacetamidomethyl)cyclohexyl]-4-benzylpiperazine 7b (13 g, 3 3 mmol) was dissolved in MeOH (192 mL) and the solution was degassed with nitrogen for 5 minutes. To the reaction flask, 10% by weight Pd on carbon (5 g) was added along with ammonium formate (6.2 g, 99 mmol). The reaction was allowed to stir at 65° C. for 2 hours. The reaction was then cooled to room temperature, filtered through celite, washed with degassed methanol, and solvent was removed under vacuum. The resulting residue was dissolved in dichloromethane (150 mL) and washed with sat. NaHCO₃ (3×150 mL) followed by washing with sat. NaCl solution (1×200 mL). The organic layer was then dried over anhydrous MgSO₄, filtered, and solvent was removed under vacuum. The deprotected piperazine was obtained as a clear oil in 86% yield without further purification. This deprotected piperazine intermediate (2.93 g, 10 mmol) was then added to a solution of 2R-methyl-3-(4-chlorophenyl)propionic acid (1.96 g, 9.87 mmol) that had been previously stirred for 1 hour at room temperature in DMF (42 mL) with HBTU (3.7 g, 9.87 mmol) and diisopropylethylamine (3.4 mL, 19.7 mmol). The reaction mixture was then allowed to stir for an additional 8 hours at room temperture. The reaction was then diluted with ethyl acetate (200 mL) and washed with washed with sat. NaHCO₃ (3×150 mL) followed by washing with sat. NaCl solution (1×200 mL). The organic layer was then dried over anhydrous Na₂SO₄, filtered, and solvent was removed under vacuum. The residue was purified by column chromatography on silica using 60% ethyl acetate/hexanes as the eluent. 1-[1-(Trifluoroacetamidomethyl)cyclohexyl]-4-[2R-methyl-3-(4-chlorophenyl)propionyl]piperazine was obtained as a clear oil in 54% yield (5.4 mmol).

Following the same procedure 1-[1-(trifluoroacetamidomethyl)cyclohexyl]-4-[2-methyl-3-(4-chloro-2-methoxyphenyl)propionyl]piperazine and 1-[1-(trifluoro-acetamidomethyl)cyclohexyl]-4-[2R-(1-pyrrolidinonyl)-3-(2,4-dichlorophenyl)propionyl]-piperazine were produced.

### Step 7D: 1-[1-(Aminomethyl)cyclohexyl]-4-[2R-methyl-3-(4-chlorophenyl)propionyl]-piperazine 7-1

1-[1-(Trifluoroacetamidomethyl)cyclohexyl]-4-[2R-methyl-3-(4-chlorophenyl) propionyl]-piperazine (3.5 mmol) was dissolved in a MeOH (50 mL)/ H₂O (4 mL) mixture along with K₂CO₃ (11.8 g) and the reaction was allowed to stir at 65° C. for 8 hours. The reaction mixture was then cooled to room temperature and diluted with dichloromethane (150 mL). The mixture was then washed with H₂O (3×100 mL) followed by sat. NaCl solution (1×150 mL). The organic layer was then dried over anhydrous MgSO₄, filtered, and solvent was removed under vacuum. 1-[1-(Aminomethyl)cyclohexyl]-4-[2-methyl-3-(4-chlorophenyl)propionyl]piperazine 7-1 was obtained as a clear yellow oil in 86% yield, which was used without further purification.

### Step 7E: 1-[2R-methyl-3-(4-chlorophenyl)propionyl-4-[1-(phenylacetamido)methyl-cyclohexylpiperazine 7-2

In a 4 mL reaction vial, a 1 mL aliquot of a 0.1M 1-[1-(aminomethyl)cyclohexyl]-4-[2R-methyl-3-(4-chlorophenyl) propionyl]piperazine 7-1 THF stock solution was added along with Et₃N (14 uL, 0.1 mmol). To the reaction vial, phenylacetyl chloride (13.2 uL, 0.1 mmol) was added and the reaction was allowed to stir at room temperature for 8 hours. The solvent was then removed by evaporation under a stream on nitrogen and the residue was dissolved in 2 mL of dichloromethane/TFA (1:1). The reaction mixture was allowed to stir at room temperature for 15 minutes then evaporated to dryness. The residue was then dissolved in 1 mL of methanol and the crude product was purified by preparative HPLC to give 1-[2R-methyl-3-(4-chlorophenyl)propionyl-4-[1-(phenylacetamido)methyl]-cyclohexylpiperazine 7-2 as a TFA salt. MS: 497 (M+H).

By the general procedures set forth above, the following compounds were also made.

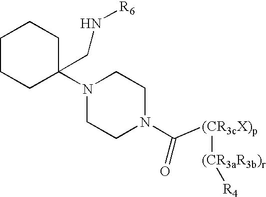

| Ex. # | —C(═O)—(CR_{3c}X)ₚ(CR₃ₐR_{3b})ᵣ—R4 | —R₆ | MS | MW |
|---|---|---|---|---|
| 7-1 | 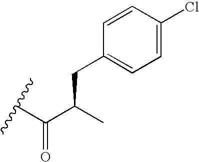 | —H | 377.9 | 378.0 |

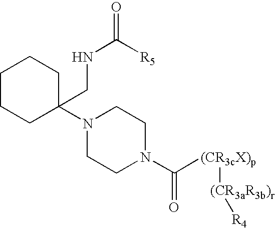

| Ex. # | —C(═O)—(CR_{3c}X)ₚ(CR₃ₐR_{3b})ᵣ—R4 | —C(═O)R₅ | MS | MW |
|---|---|---|---|---|
| 7-2 | 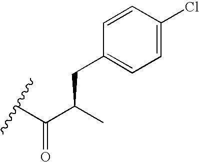 | 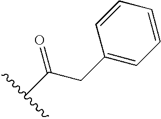 | 495.8 | 496.1 |
| 7-3 | 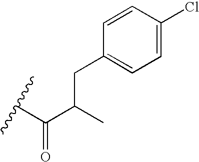 | 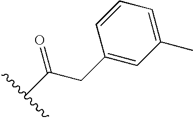 | 510.3 | 510.1 |
| 7-4 | 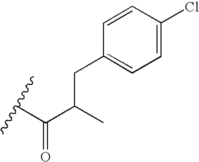 | 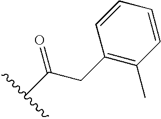 | 510.3 | 510.1 |
| 7-5 | 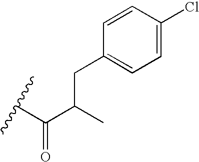 | 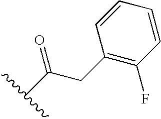 | 514.3 | 514.1 |
| 7-6 | 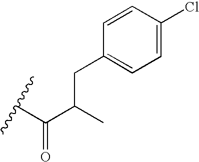 | 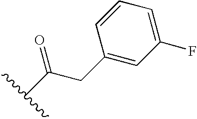 | 514.2 | 514.1 |
| 7-7 | 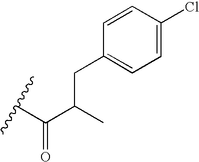 | 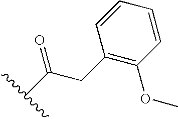 | 526.3 | 526.1 |
| 7-8 | 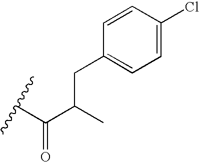 | 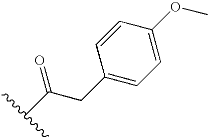 | 526.3 | 526.1 |
| 7-9 | 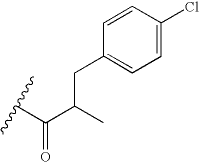 | 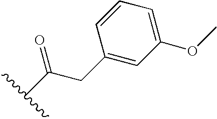 | 526.3 | 526.1 |
| 7-10 | 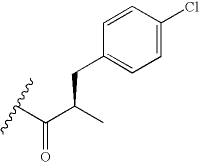 | 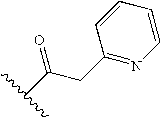 | 496.8 | 497.1 |
| 7-11 | 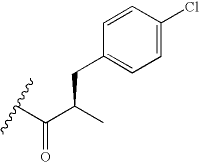 | 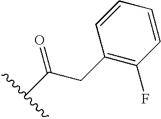 | 513.8 | 514.1 |
| 7-12 | 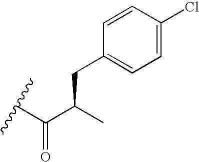 | 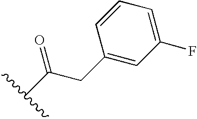 | 513.8 | 514.1 |
| 7-13 | 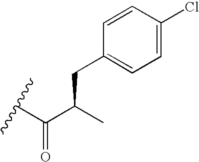 | 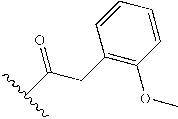 | 525.8 | 526.1 |
| 7-14 | 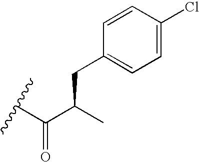 | 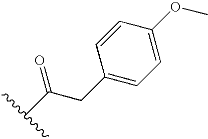 | 525.8 | 526.1 |
| 7-15 | 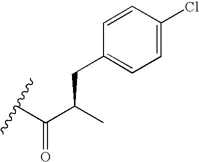 | 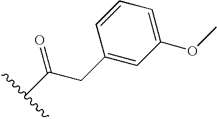 | 525.8 | 526.1 |
| 7-16 | 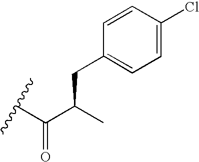 | 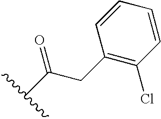 | 529.7 | 530.5 |
| 7-17 | 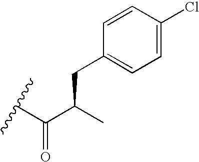 | 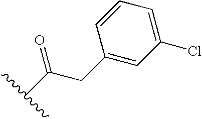 | 529.8 | 530.5 |
| 7-18 | 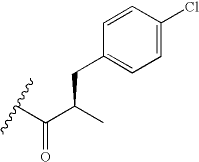 | 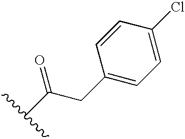 | 529.8 | 530.5 |
| 7-19 | 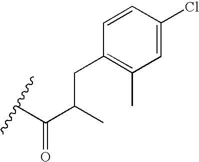 | 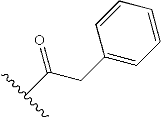 | 510.3 | 510.1 |
| 7-20 | 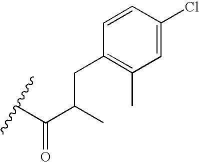 | 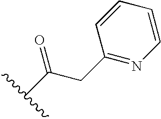 | 511.3 | 511.1 |
| 7-21 | 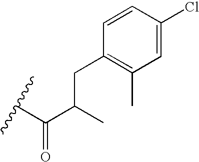 | 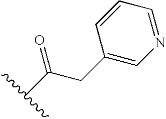 | 511.2 | 511.1 |
| 7-22 | 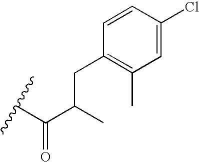 | 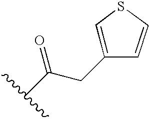 | 516.3 | 516.1 |
| 7-23 | 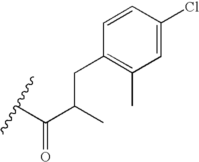 | 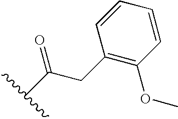 | 540.3 | 540.1 |
| 7-24 | 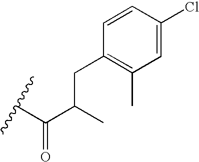 | 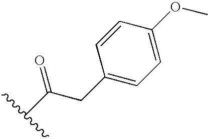 | 540.3 | 540.1 |
| 7-25 | 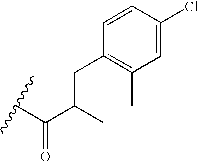 | 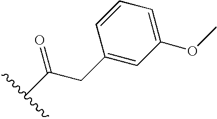 | 540.3 | 540.1 |
| 7-26 | 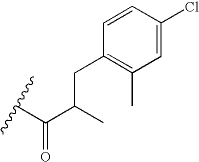 | 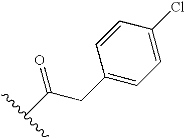 | 544.3 | 544.6 |
| 7-27 | 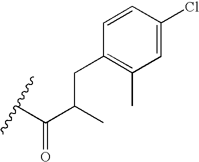 | 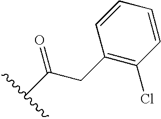 | 544.2 | 544.6 |
| 7-28 | 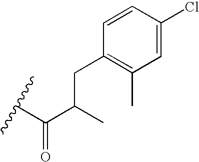 | 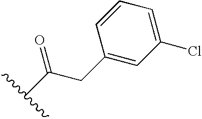 | 544.2 | 544.6 |
| 7-29 | 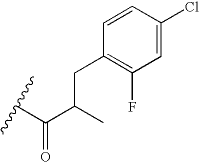 | 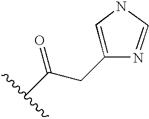 | 504.2 | 504.0 |
| 7-30 | 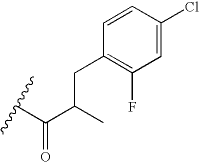 | 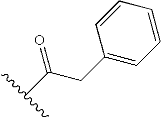 | 514.2 | 514.1 |
| 7-31 | 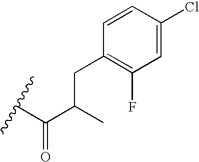 | 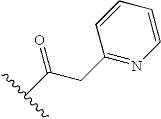 | 515.2 | 515.1 |
| 7-32 | 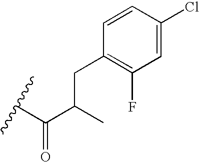 | 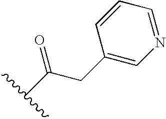 | 515.3 | 515.1 |
| 7-33 | 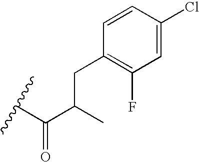 | 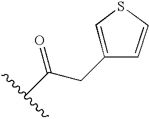 | 520.2 | 520.1 |
| 7-34 | 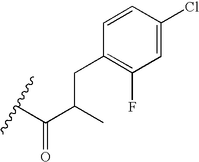 | 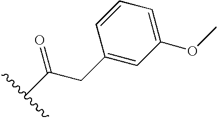 | 544.2 | 544.1 |
| 7-35 | 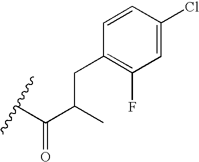 | 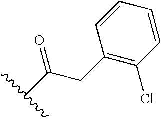 | 548.2 | 548.5 |
| 7-36 | 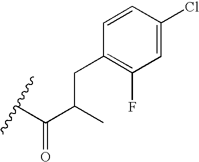 | 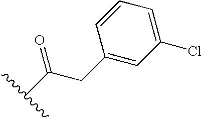 | 548.2 | 548.5 |
| 7-37 | 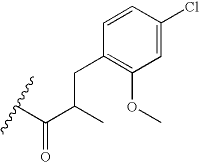 | 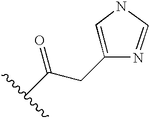 | 515.8 | 516.1 |
| 7-38 | 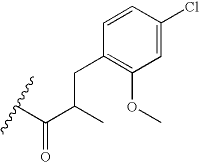 | 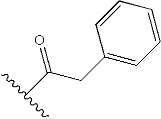 | 525.8 | 526.1 |
| 7-39 | 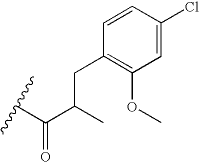 | 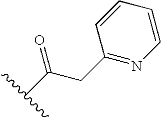 | 526.8 | 527.1 |
| 7-40 | 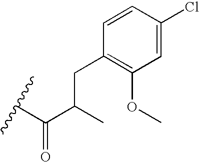 | 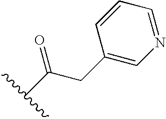 | 526.8 | 527.1 |
| 7-41 | 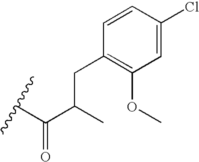 | 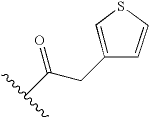 | 531.7 | 532.1 |
| 7-42 | 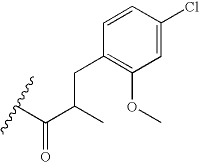 | 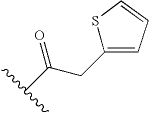 | 531.7 | 532.1 |
| 7-43 | 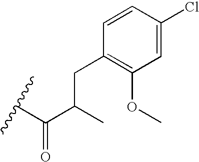 | 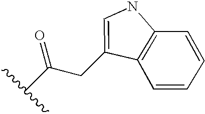 | 564.8 | 565.2 |
| 7-44 | 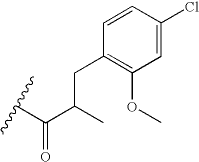 | 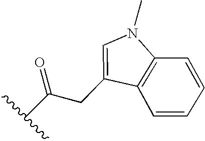 | 578.8 | 579.2 |
| 7-45 | 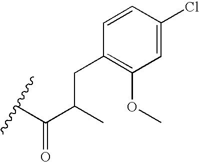 | 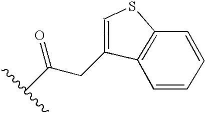 | 581.7 | 582.2 |
| 7-46 | 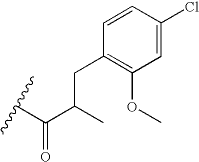 | 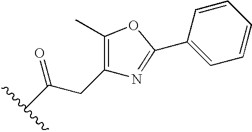 | 606.7 | 607.2 |
| 7-47 | 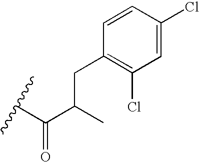 | 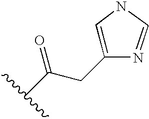 | 520.2 | 520.5 |
| 7-48 | 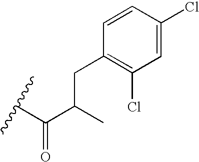 | 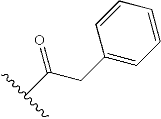 | 530.2 | 530.5 |
| 7-49 | 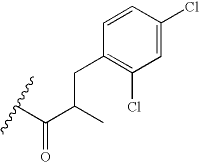 | 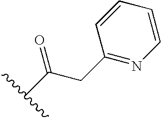 | 531.2 | 531.5 |
| 7-50 | 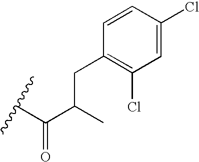 | 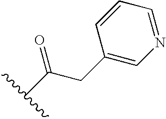 | 531.2 | 531.5 |
| 7-51 | 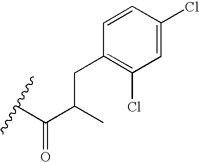 | 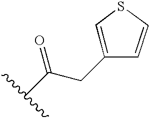 | 536.2 | 536.6 |
| 7-52 | 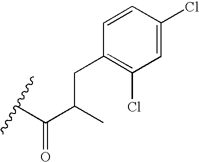 | 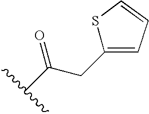 | 536.2 | 536.6 |
| 7-53 | 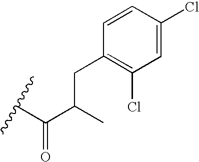 | 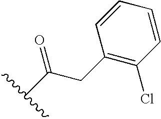 | 566.2 | 565.0 |
| 7-54 | 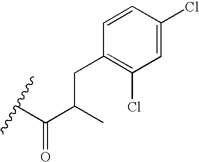 | 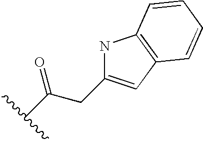 | 569.2 | 569.6 |
| 7-55 | 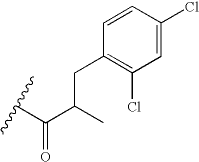 | 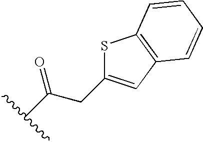 | 586.2 | 586.6 |
| 7-56 | 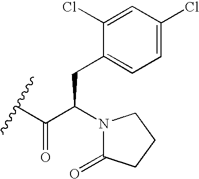 | 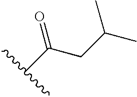 | 564.7 | 565.6 |
| 7-57 | 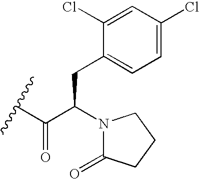 | 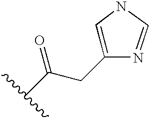 | 588.7 | 589.6 |
| 7-58 | 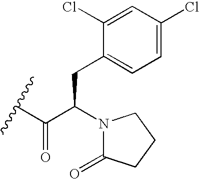 | 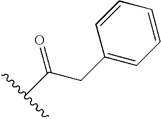 | 598.7 | 599.6 |
| 7-59 | 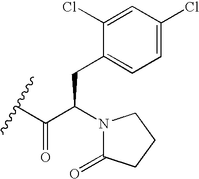 | 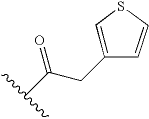 | 604.6 | 605.6 |
| 7-60 | 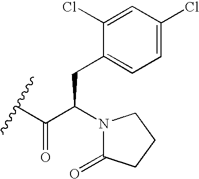 | 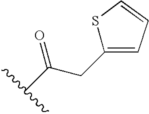 | 604.7 | 605.6 |
| 7-61 | 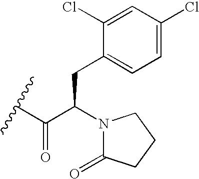 | 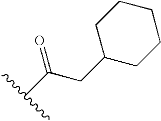 | 604.7 | 605.6 |
| 7-62 | 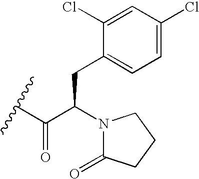 | 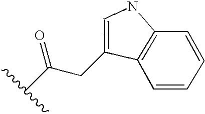 | 637.7 | 638.6 |
| 7-63 | 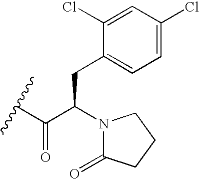 | 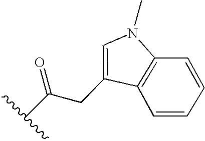 | 651.7 | 652.7 |
| 7-64 | 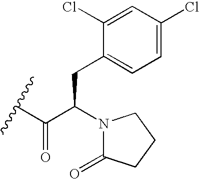 | 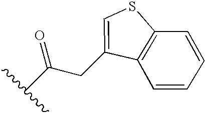 | 654.6 | 655.7 |
| 7-65 | 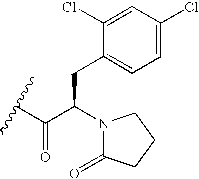 | 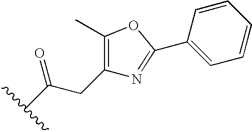 | 679.6 | 680.7 |

### Example 8

### 1-[2-METHYL-3-(4-CHLOROPHENYL)PROPIONYL)-4-[1-(N-BENZYLAMINO)METHYL]CYCLOHEXYLPIPERAZINE

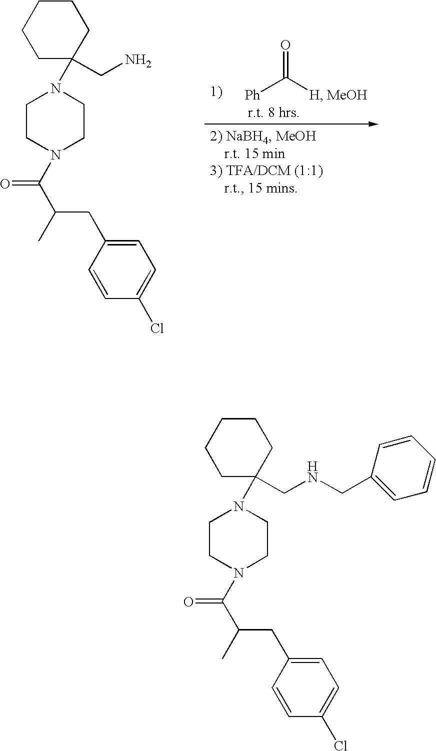

In a 4 mL reaction vial, a 1 mL aliquot of the 0.1M 1-[2-methyl-3-(4-chlorophenyl)propionyl-4-(1-aminomethyl)cyclohexylpiperazine (compound 7-1) MeOH stock solution was added along with benzaldehyde (10 uL, 0.1 mmol). The reaction mixture was stirred at room temperature for 8 hours. Then, to the reaction vial, NaBH₄ (6.1 mg, 0.16 mmol) was added and the reaction was stirred at room temperature for an additional 15 minutes. The reaction mix was then quenched with 1 mL of 1N NaOH and the product was extracted with ether. The ethereal extract was then concentrated under a stream on nitrogen and the residue was then dissolved in 1 mL of methanol and was purified by preparative HPLC. 1-[2-Methyl-3-(4-chlorophenyl)propionyl-4-[1-(N-benzylamino)methyl]cyclohexylpiperazine 8-1 was obtained as the TFA salt in 52% overall yield. MS: calc. for C₂₈H₃₈ClN₃O: 468; found: 469 (M+H).

By the general procedures set forth above, the following compounds were also made.

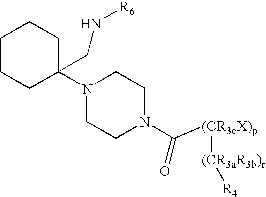

| Ex. # | —C(═O)—(CR_{3c}X)ₚ(CR₃ₐR_{3b})ᵣ—R4 | —R₆ | MS | MW |
|---|---|---|---|---|
| 8-1 | 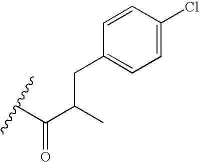 | 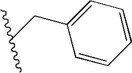 | 467.8 | 468.1 |
| 8-2 | 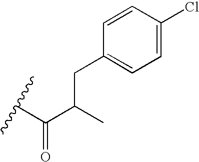 | 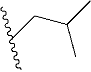 | 434.3 | 434.1 |
| 8-3 | 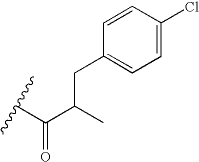 | 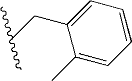 | 481.8 | 482.1 |
| 8-4 | 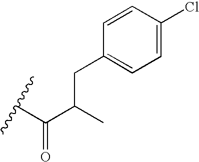 | 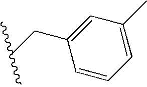 | 481.8 | 482.1 |
| 8-5 | 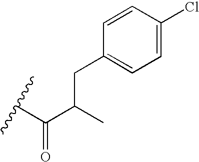 | 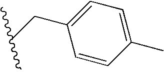 | 481.8 | 482.1 |
| 8-6 | 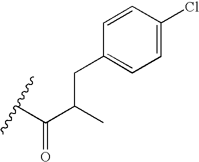 | 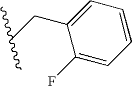 | 485.8 | 486.1 |
| 8-7 | 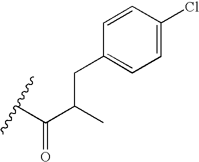 | 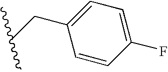 | 485.8 | 486.1 |
| 8-8 | 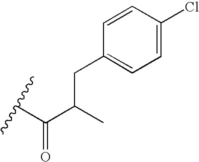 | 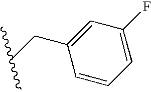 | 485.8 | 486.1 |
| 8-9 | 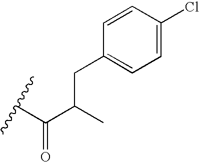 | 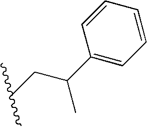 | 495.8 | 496.1 |
| 8-10 | 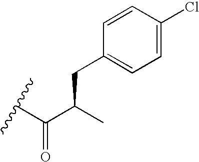 | 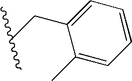 | 481.8 | 482.1 |
| 8-11 | 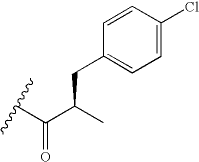 | 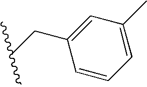 | 481.9 | 482.1 |
| 8-12 | 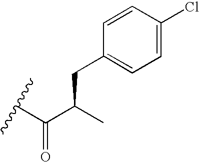 | 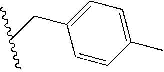 | 481.9 | 482.1 |
| 8-13 | 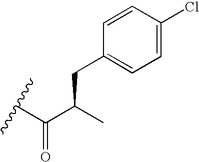 | 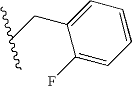 | 485.8 | 486.1 |
| 8-14 | 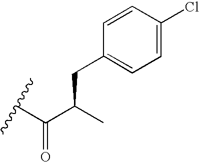 | 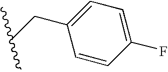 | 485.8 | 486.1 |
| 8-15 | 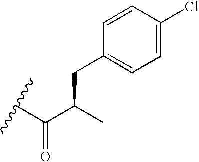 | 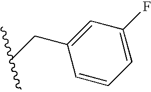 | 485.8 | 486.1 |
| 8-16 | 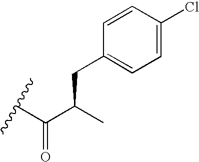 | 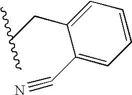 | 492.8 | 493.1 |
| 8-17 | 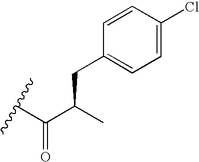 | 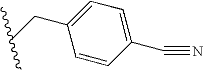 | 492.8 | 493.1 |
| 8-18 | 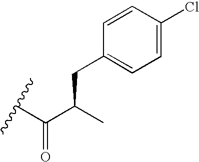 | 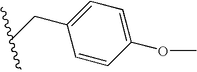 | 497.9 | 498.1 |
| 8-19 | 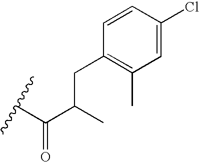 | 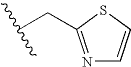 | 488.8 | 489.1 |
| 8-20 | 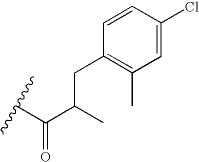 | 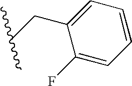 | 499.8 | 500.1 |
| 8-21 | 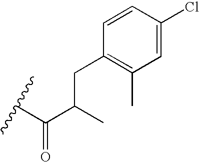 | 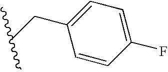 | 499.8 | 500.1 |
| 8-22 | 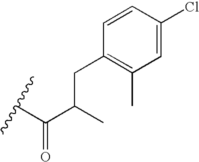 | 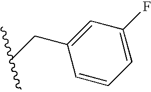 | 499.8 | 500.1 |
| 8-23 | 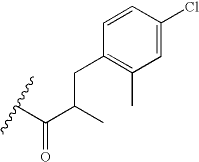 | 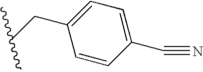 | 506.8 | 507.1 |
| 8-24 | 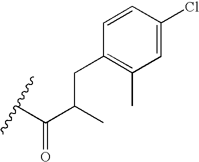 | 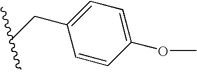 | 511.8 | 512.1 |
| 8-25 | 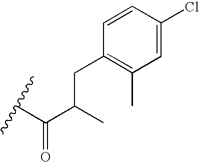 | 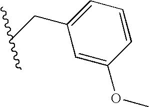 | 511.8 | 512.1 |
| 8-26 | 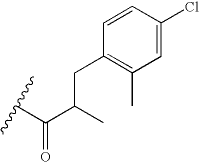 | 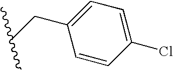 | 515.8 | 516.6 |
| 8-27 | 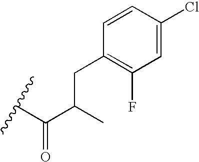 | 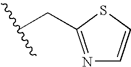 | 492.7 | 493.1 |
| 8-28 | 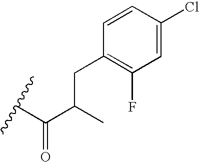 | 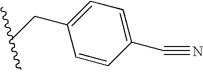 | 510.8 | 511.1 |
| 8-29 | 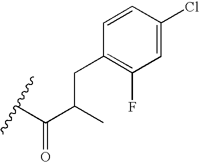 | 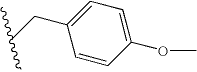 | 515.8 | 516.1 |
| 8-30 | 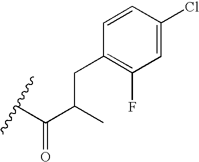 | 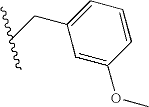 | 515.8 | 516.1 |
| 8-31 | 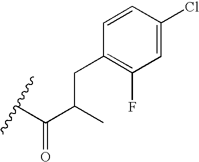 | 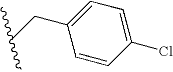 | 519.7 | 520.5 |
| 8-32 | 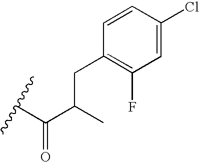 | 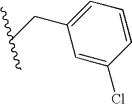 | 519.7 | 520.5 |
| 8-33 | 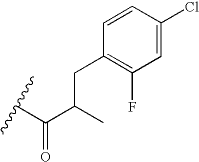 | 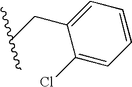 | 519.7 | 520.5 |
| 8-34 | 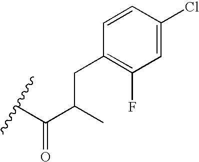 | 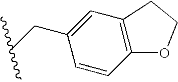 | 527.8 | 528.1 |
| 8-35 | 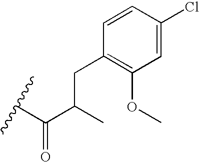 | 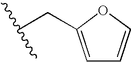 | 487.8 | 488.1 |
| 8-36 | 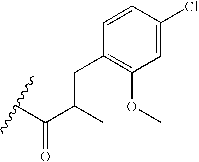 | 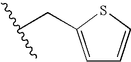 | 503.8 | 504.1 |
| 8-37 | 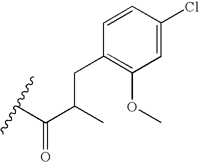 | 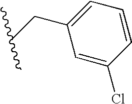 | 531.8 | 532.6 |
| 8-38 | 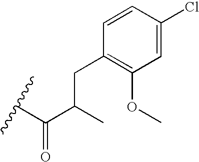 | 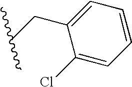 | 531.7 | 532.6 |
| 8-39 | 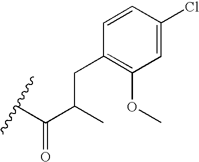 | 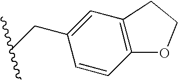 | 539.8 | 540.1 |
| 8-40 | 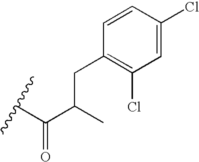 | 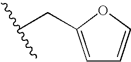 | 491.8 | 492.5 |
| 8-41 | 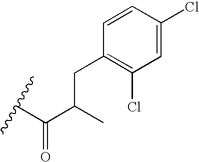 | 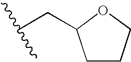 | 495.8 | 496.5 |
| 8-42 | 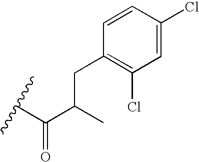 | 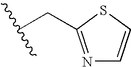 | 508.7 | 509.5 |
| 8-43 | 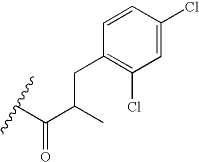 | 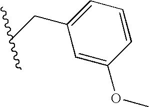 | 531.7 | 532.6 |
| 8-44 | 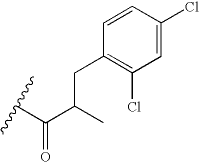 | 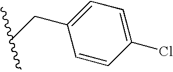 | 537.7 | 537.0 |
| 8-45 | 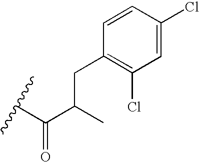 | 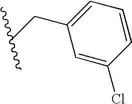 | 535.7 | 537.0 |
| 8-46 | 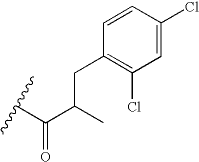 | 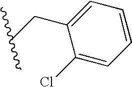 | 535.7 | 537.0 |
| 8-47 | 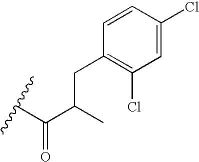 | 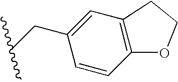 | 543.7 | 544.6 |
| 8-48 | 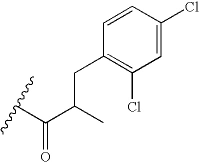 | 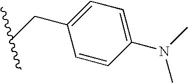 | 544.8 | 545.6 |
| 8-49 | 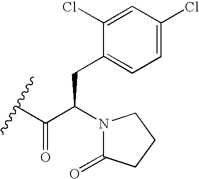 | 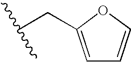 | 560.7 | 561.6 |
| 8-50 | 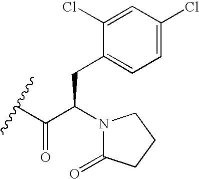 | 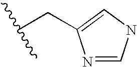 | 560.7 | 561.6 |
| 8-51 | 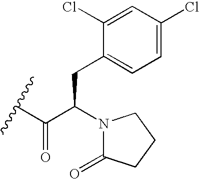 | 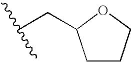 | 564.7 | 565.6 |
| 8-52 | 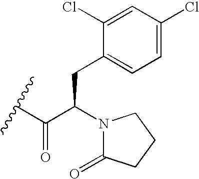 | 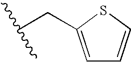 | 576.7 | 577.6 |
| 8-53 | 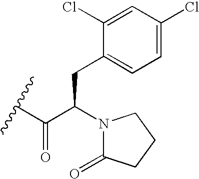 | 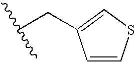 | 576.7 | 577.6 |
| 8-54 | 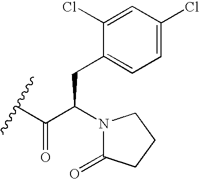 | 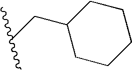 | 576.8 | 577.6 |
| 8-55 | 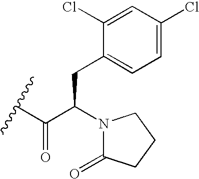 | 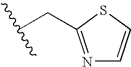 | 577.7 | 578.6 |
| 8-56 | 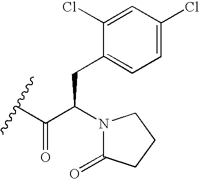 | 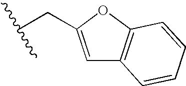 | 610.7 | 611.6 |

### Example 9

### 1-[2-METHYL-3-(4-CHLOROPHENYL)PROPIONYL-4-[1-(N-ISOBUTYL-(METHYLAMINOACETAMIDO)METHYL]CYCLOHEXYLPIPERAZINE

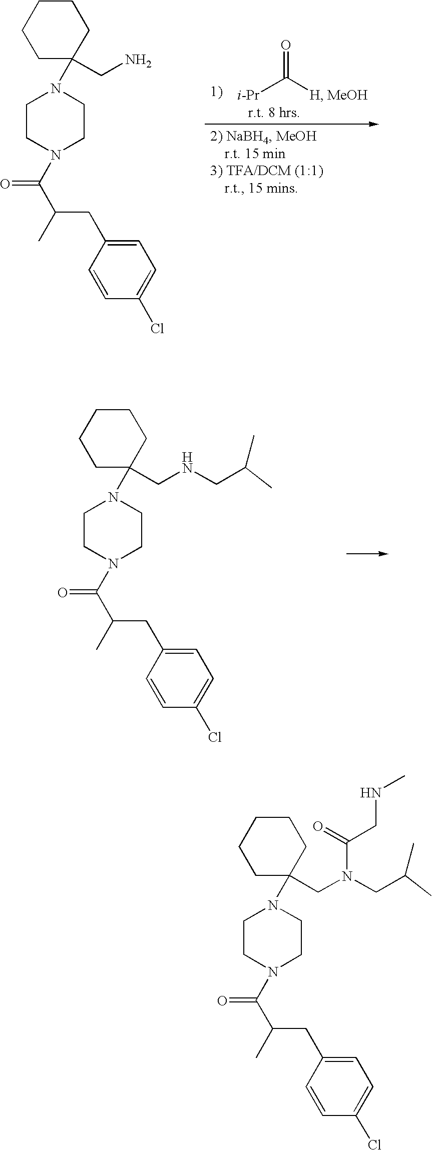

### Step 9A: 1-[2-Methyl-3-(4-chlorophenyl)propionyl-4-[1-(N-isobutylmethyl)]cyclohexyl-piperazine 9a

In a 4 mL reaction vial, a 1 mL aliquot of the 0.1M 1-[2-methyl-3-(4-chlorophenyl)propionyl-4-(1-aminomethyl)cyclohexylpiperazine 7-1 MeOH stock solution was added along with isobutyraldehyde (0.1 mmol). The reaction mixture was stirred at room temperature for 8 hours. NaBH₄ (6.1 mg, 0.16 mmol) was added and the reaction was stirred at room temperature for an additional 15 minutes. The reaction mixture was then quenched with 1 mL of IN NaOH and the product was extracted with ether. The ethereal extract was then concentrated under a stream on nitrogen and the residue was then dissolved in 1 mL of methanol and was purified by preparative HPLC. 1-[2-Methyl-3-(4-chlorophenyl)propionyl-4-[1-(N-isobutylmethyl)]cyclohexylpiperazine 9a was obtained as the TFA salt in 52% overall yield. MS: calc. for C₂₅H₄₀ClN₃O: 434; found: 435 (M+H).

### Step 9B: 1-[2-Methyl-3-(4-chlorophenyl)propionyl-4-{1-[N-isobutyl-(methylamino-acetamido)methyl]}cyclohexylpiperazine 9-1

In a 4 mL reaction vial, a 1 mL aliquot of a 0.1M 1-[1-(aminomethyl)cyclohexyl]-4-[2-methyl-3-(4-chlorophenyl)]piperazine 9a THF stock solution was added along with Et₃N (14 uL, 0.1 mmol). To the reaction vial, N-Boc-sarcosin (0.1 mmol) was added and the reaction mixture was stirred at room temperature for 8 hours. The solvent was then removed by evaporation under a stream on nitrogen and the residue was dissolved in 2 mL of dichloromethane/TFA (1:1). The reaction mixture was stirred at room temperature for 15 minutes then was evaporated to dryness. The residue was dissolved in 1 mL of methanol and the crude product was purified by preparative HPLC to give 1-[2-methyl-3-(4-chlorophenyl)propionyl-4-{1-[N-isobutyl-(methylaminoacetamido)methyl]}-cyclohexyl-piperazine 9-1 as a TFA salt.

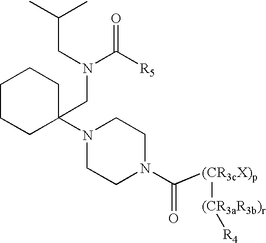

| Ex. # | —C(═O)—(CR_{3c}X)ₚ—(CR₃ₐR_{3b})ᵣ—R₄ | —C(═O)R₅ | MS | MW |
|---|---|---|---|---|
| 9-1 | 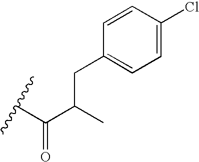 | 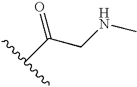 | 505.3 | 505.1 |
| 9-2 | 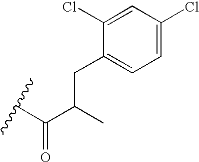 | 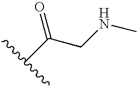 | 539.2 | 539.6 |
| 9-3 | 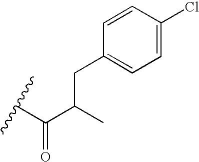 | 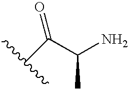 | 505.3 | 505.1 |
| 9-4 | 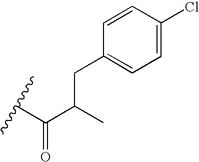 | 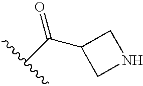 | 517.3 | 517.2 |

### Example 10

### 1-[(2-METHYL-3-(2-METHYL-4-CHLOROPHENYL)PROPIONYL]-4-[2-(METHOXYCARBONYLMETHYL)CYCLOHEPTYL]PIPERAZINE

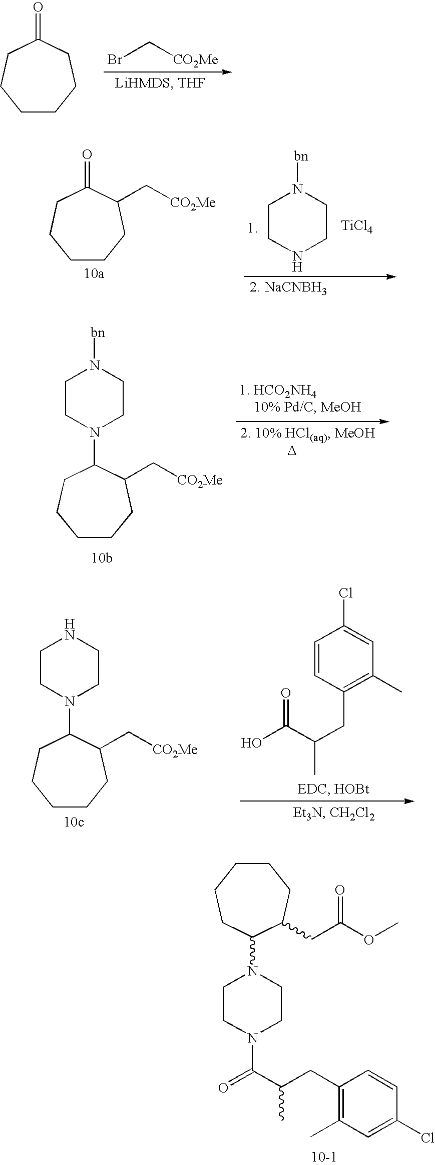

### Step 10A: 2-(methoxycarbonylmethyl)cycloheptanone 10a

To a solution of cycloheptanone (5.53 mL, 46.9 mmol) in THF (200 mL) at −78° C. was added LiHMDS (1.0 M in pentane, 51.6 mL, 51.6 mmol). After 0.5 h, methyl bromoacetate (4.9 mL, 51.6 mmol) was added to the reaction dropwise. The reaction was stirred at the same temperature for 2 h and then was quenched with saturated aqueous ammonium chloride solution (200 mL). The mixture was warmed to room temperature then extracted with diethyl ether. The combined organic layers were washed with brine, dried (Na₂SO₄), filtered and evaporated. Silica-gel chromatography (2:1 hexanes: ethyl acetate) afforded the ketone 10a (5.00 g, 27.1 mmol) in 58% yield. ¹H NMR (300 MHz, CDCl₃) δ 3.66 (s, 3H), 3.17-3.06 (m, 1H), 2.83 (dd, J=16.8, 8.4 Hz, 1H), 2.65 (dtd, J=16.2, 4.7, 1.5 Hz, 1H), 2.45 (J=15.6, 10.8, 4.8 Hz, 1H), 2.30 (dd, J=16.8, 5.7 Hz, 1H), 1.99-1.20 (m, 8H).

### Step 10B: 1-Benzyl-4-[2-(methoxycarbonylmethyl)cycloheptyl]piperazine 10b

2-(Methoxycarbonylmethyl)cycloheptanone 10a (4.0 g, 21.7 mmol) and benzyl piperazine (6.93 mL, 43.4 mmol) were dissolved in dichloromethane (110 mL) and then cooled to 0° C. TiCl₄ (1M in dichloromethane, 22.8 mL) was added dropwise through an addition funnel. After 0.5 h, the reaction was warmed to room temperature and was stirred for another 2.5 hours. Sodiumcyanoborohydride (5.47 g, 86.8 mmol) was added to the mixture and the reaction was stirred for 16 hours. LC/MS showed two isomeric products were formed in a 4:1 ratio. Wate (10 mL) was added and the mixture was stirred for 20 minutes and was filtered. The filtrate was concentrated and the products were purified by flash column chromatography (Hex:EtOAC 10:1 to 1:1). The cis and trans isomers of 10b (1.01 g and 0.57 g) were isolated, giving a combined yield of 21%, LC/MS m/z 345.3 (MH⁺).

### Step 10C: 4-[2-(Methoxycarbonylmethyl)cycloheptyl]piperazine 10c

To 1-benzyl-4-[2-(methoxycarbonylmethyl)cycloheptyl]piperazine 10b (1.3 g, 3.8 mmol) solution in EtOH (40 mL) was added 10% Pd/C (1 g) and the reaction was heated to reflux, then HCO₂NH₄ (710 mg, 11.4 mmol) was added to the mixture and the heating continued for 1 h. The mixture was cooled and was filtered through a pad of celite and concentrated. The formylated product (MW 282) was obtained in 600 mg quantity. It was then refluxed in 10% aqueous HCl/MeOH (1:1) mixture for 3 h, and cooled. The solution was extracted with ethyl ether then adjusted to pH 10 with 2N NaOH. The mix was extracted with EtOAc twice. The combined organic layers were washed with brine, dried over MgSO₄, filtered and concentrated to afford 204 mg of 4-[2-(methoxycarbonylmethyl)cycloheptyl]piperazine 10c (0.8 mmol) with 21% yield over two steps. GC/MS m/z 254 (M⁺).

### Step 10D: 1-[2-Methyl-3-(2-methyl-4-chlorophenyl)-4-[2-(methoxycarbonylmethyl)-cycloheptyl]piperazine 10-1

To the solution of 4-[2-(methoxycarbonylmethyl)cycloheptyl]piperazine 10c (26 mg, 0.1 mmol,) and 2-methyl-3-(2-methyl-4-chlorophenyl)propionic acid (26 mg, 0. 12 mmol) in 0.5 mL of dichloromethane was added 1-(3-dimethylaminopropyl)-3-ethylcarbodiimide hydrochloride (EDC, 23 mg, 0.12 mmol) and 1-hydroxybenzotriazole (HOBt, 16 mg, 0.12 mmol). Triethylamine (33 μl, 0.24 mmol) was added and the mixture was stirred at room temperature for 16 h. Saturated NaHCO₃ solution (1 mL) was added. The aqueous layer was extracted with dichloromethane twice and the organic layer was filtered through a Na₂SO₄ pad, and concentrated. The residue was purified by preparative LC/MS to afford 25 mg of 1-[2-methyl-3-(2-methyl-4-chlorophenyl)-4-[2-(methoxycarbonylmethyl)cycloheptyl]piperazine 10-1 as the TFA salt in 44% yield. LC/MS m/z 449.0 (MH⁺).

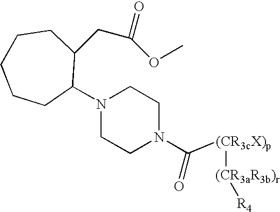

| Ex. # | —C(═O)—(CR_{3c}X)ₚ—(CR₃ₐR_{3b})ᵣ—R₄ | MS | MW |
|---|---|---|---|
| 10-1 | 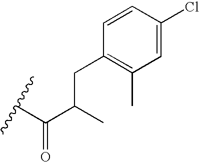 | 449.0 | 449.0 |
| 10-2 | 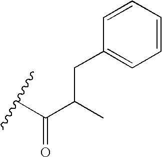 | 401.1 | 400.6 |
| 10-3 | 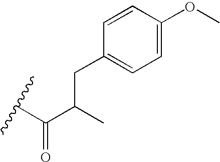 | 431.1 | 430.6 |
| 10-4 | 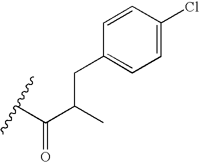 | 449.0 | 435.0 |
| 10-5 | 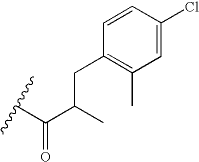 | 429.1 | 428.6 |
| 10-6 | 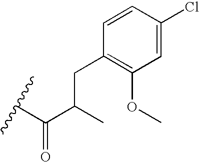 | 465.0 | 465.0 |
| 10-7 | 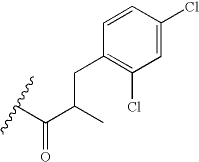 | 469.0 | 469.5 |

### Example 11

### TRANS-2-{4-[2-METHYL-3-(4-CHLOROPHENYL)PROPIONYL]PIPERAZIN-1-YL}-CYCLOHEXANECARBOXYLIC ACID ETHYL ESTER

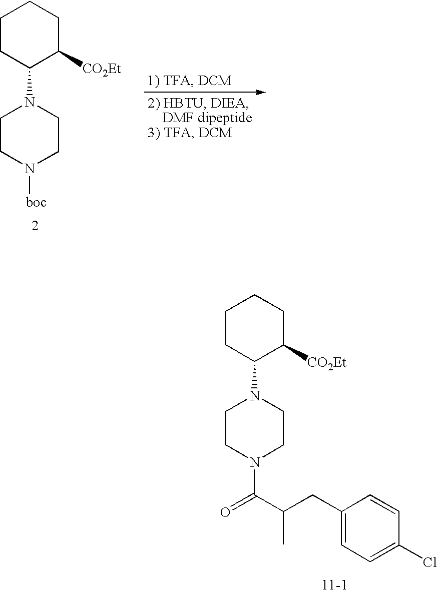

### Step 11A: trans-2-{4-[2-Methyl-3-(4-chlorophenyl)propionyl]piperazin-1-yl}-cyclohexanecarboxylic acid ethyl ester 11-1

trans-4-(2-Ethoxycarbonyl-cyclohexyl)-piperazine-1-carboxylic acid tert-butyl ester (compound 2 from Example 1, 136 mg, 0.4 mmol) was dissolved in dichloromethane (2 mL) and to that solution, trifluoroacetic acid (1 mL) was added. The resulting solution was stirred at room temperature for 1 h. The volatiles were removed in vacuo. The residue was then dissolved in DMF (1 mL) and treated with diisopropylethyl amine (140 μL, 0.80 mmol).

In a separate flask, a solution containing 2-methyl-3-(4-chlorophenyl)propionic acid (87 mg, 0.44 mmol) and diisopropylethyl amine (140 μL, 0.80 mmol) in DMF (2 mL), was treated with HBTU (200 mg, 0.52 mmol). The resulting solution was stirred at room temperature, under N₂, for 30 minutes. The DMF solution containing the deprotected amine was added and the resulting mixture was stirred for 16 h at room temperature. The mixture was diluted with EtOAc (30 mL) and was washed with 0.1 N HCl and then with saturated NaHCO₃. The organics were washed with brine, dried over anhydrous MgSO₄ and filtered. Evaporation gave a residue which was purified by preparative HPLC/MS to give trans-2-{4-[2-methyl-3-(4-chlorophenyl)propionyl]piperazin-1-yl}-cyclohexanecarboxylic acid ethyl ester 11-1 (0.17 mmol, 42 %). LCMS m/z 422 (M⁺+1).

By the general procedures set forth above, the following compounds were also made.

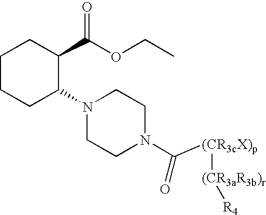

| Ex. # | —C(═O)—(CR_{3c}X)ₚ—(CR₃ₐR_{3b})ᵣR₄ | MS | MW |
|---|---|---|---|
| 11-1 | 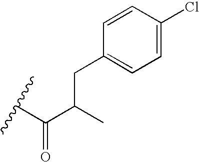 | 420.8 | 421.0 |
| 11-2 | 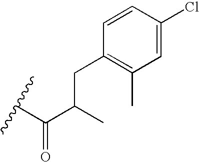 | 434.8 | 435.0 |
| 11-3 | 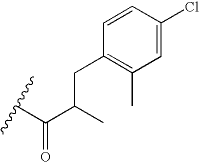 | 434.8 | 435.0 |
| 11-4 | 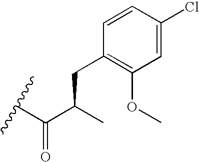 | 450.8 | 451.0 |
| 11-5 | 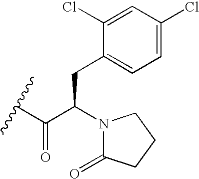 | 489.8 | 490.0 |
| 11-6 | 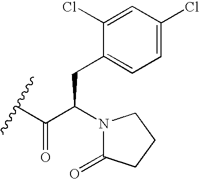 | 524.1 | 524.5 |

### Example 12

### TRANS-2-{4-[2-(2-Oxo-1-IMIDAZOLIDINYL)-3-(2,4-DICHLOROPHENYL)PROPIONYL]PIPERAZIN-1-YL}-CYCLOHEXANECARBOXYLIC ACID ETHYL ESTER

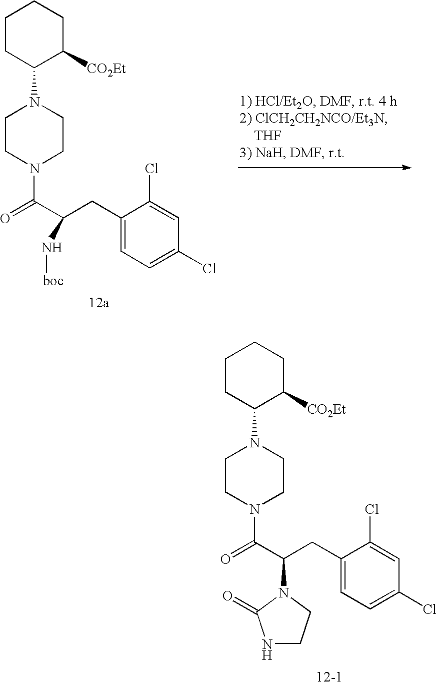

trans-2-{4-[2-(N-Boc-amino)-3-(2,4-dichlorophenyl)propionyl]piperazin-1-yl}-cyclohexanecarboxylic acid ethyl ester (2.39 mmol) 12a was dissolved in dichloromethane (15 mL) along with 10 mL of 2M HCl in ether solution. The reaction mixture was allowed to stir at room temperature for 4 hours then solvent was removed in vacuo. The deprotected amine was recovered as the HCl salt in 88% yield (0.97 g, 2.1 mmol) and was then dissolved in THF (8 mL) along with 2-chloroethyl isocyanate (182 uL, 2.1 mmol) and Et₃N (585 uL, 4.21 mmol). The reaction mixture was stirred at room temperature for 8 hours then was washed with saturated NaHCO₃ solution (3×15 mL) and saturated NaCl solution (15 mL). The organic layer was separated, dried over anhydrous MgSO₄, filtered, and solvent was removed in vacuo. The residue was purified by column chromatography on silica using 50% ethyl acetate/hexanes as the eluent to give the urea intermediate in 74% overall yield.

The urea intermediate (1.77 mmol) was dissolved in DMF (4 mL) and stirred at room temperature. To the reaction mixture, NaH (89 mg, 2.22 mmol) was added in small portions over a period of 30 minutes. After the addition, the reaction mixture was stirred at room temperature for an additional 1.5 hours then was quenched with water (10 mL). The reaction mixture was extracted with ethyl acetate (3×10 mL). The organic layers were combined, dried over anhydrous MgSO₄, filtered, and the solvent was removed in vacuo. The crude product was purified by column chromatography on silica using 85% ethyl acetate/hexanes as the eluent. trans-2-{4-[2-(2-Oxo-1-imidazolidinyl)-3-(2,4-dichlorophenyl)propionyl]piperazin-1-yl}-cyclohexanecarboxylic acid ethyl ester 12-1 was obtained in 55% yield. MS 526 (MH⁺).

### Example 13

### TRANS-2-{4-[2-(2-Oxo-3-AMINO-1-PYRROLIDINYL)-3-(2,4-DICHLOROPHENYL)PROPIONYL]PIPERAZIN-1-YL}-CYCLOHEXANECARBOXYLIC ACID ETHYL ESTER

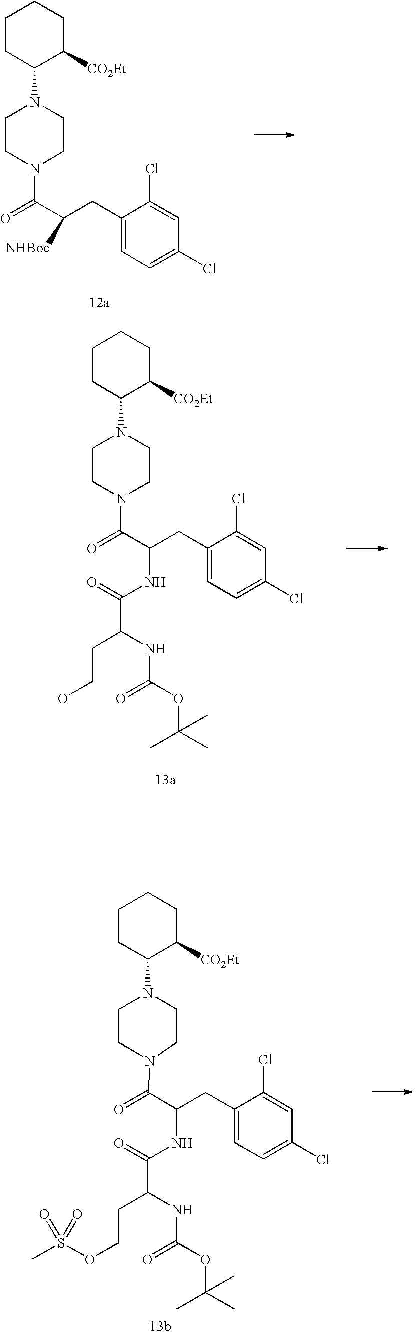

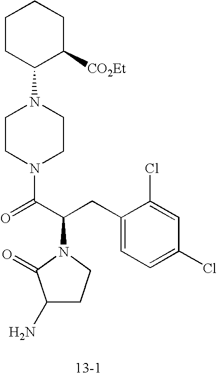

### Step 13A: trans-2-{4-[2-(2-Boc-amino-4-hydroxybutyroylamino)-3-(2,4-dichlorophenyl)-propionyl]piperazin-1-yl}-cyclohexanecarboxylic acid ethyl ester 13a

To a mixture of trans-2-{4-[2-(N-Boc-amino)-3-(2,4-dichlorophenyl) propionyl]piperazin-1-yl}-cyclohexanecarboxylic acid ethyl ester 12a (0.16 mmol) in dry methylene chloride (2 mL) under nitrogen, was added trimethylaluminium (0.17 mL, 0.33 mmol) dropwise at room temperature. The reaction mixture was then stirred for 15 minutes and a solution of tert-butyl (tetrahydro-2-oxo-3-furanyl)carbamate (32 mg, 0.16 mmol) dissolved in dry methylene chloride (2 mL) was then added dropwise to the reaction at room temperature and stirred overnight. The mixture was quenched with 4 mL of 10% citric acid, partitioned between methylene chloride and potassium sodium tartrate. The organic layer was separated, dried over magnesium sulfate and then the solvent was removed in vacuo to obtain trans-2-{4-[2-(2-Boc-amino-4-hydroxybutyroylamino)-3-(2,4-dichlorophenyl)propionyl]-piperazin-1-yl}-cyclohexanecarboxylic acid ethyl ester 13a. LCMS m/z 657 (MH⁺).

### Step 13B: trans-2-{4-[2-(2-1Boc-amino-4-methanesulfonyloxybutyroylamino)-3-(2,4-dichlorophenyl)propionyl]piperazin-1-yl}-cyclohexanecarboxylic acid ethyl ester 13b

To a mixture of trans-2-{4-[2-(2-Boc-amino-4-hydroxybutyroylamino)-3-(2,4-dichlorophenyl)propionyl]piperazin-1-yl}-cyclohexanecarboxylic acid ethyl ester 13a (159 mg, 0.19 mmol) in dry methylene chloride (5 mL) was added triethylamine (55 uL, 0.38 mmol) and methanesulfonyl chloride (15 uL, 0.19 mmol) at 0° C. The mixture was allowed to stir for 2 hours, gradually warming to room temperature. The reaction was then partitioned between methylene chloride and sodium bicarbonate. The organic layer was separated, dried over magnesium sulfate, and removed in vacuo to obtain 13b as a white foam. LCMS m/z 735 (MH⁺).

### Step 13C: trans-2-{4-[2-(2-Oxo-3-amino-1-pyrrolidinyl)-3-(2,4-dichlorophenyl)propionyl]piperazin-1-yl}-cyclohexanecarboxylic acid ethyl ester 13-1

To a mixture of trans-2-{4-[2-(2-Boc-amino-4-methanesulfonyloxybutyroyl-amino)-3-(2,4-dichlorophenyl)propionyl]piperazin-1-yl}-cyclohexanecarboxylic acid ethyl ester 13b (163 mg, 0.18 mmol) in tetrahydrofuran (10 mL) was added sodium hydride (22 mg, 0.54 mmol). The reaction mix was stirred overnight, and then partitioned between methylene chloride and saturated ammonium chloride. The organic layer was separated, dried over magnesium sulfate and removed in vacuo to yield the protected intermediate. Trifluoroacetic acid (2 mL) and methylene chloride (2 mL) were added to 46 mg of the protected intermediate and the mix was stirred at room temperature for forty-five minutes. The solvent was then removed in vacuo to give a residue which was purified by preparative liquid chromatography to give trans-2-{4-[2-(2-oxo-3-amino-1-pyrrolidinyl)-3-(2,4-dichlorophenyl)-propionyl]piperazin-1-yl}-cyclohexanecarboxylic acid ethyl ester 13-1 as a clear oil (35mg). LCMS m/z 540 (MH⁺).

### Example 14

### TRANS-2-{4-[2-(2-Oxo-1-PIPERAZINYL)-3-(4-CHLOROPHENYL)PROPIONYL]PIPERAZIN-1-YL}-CYCLOHEXANECARBOXYLIC ACID ETHYL ESTER

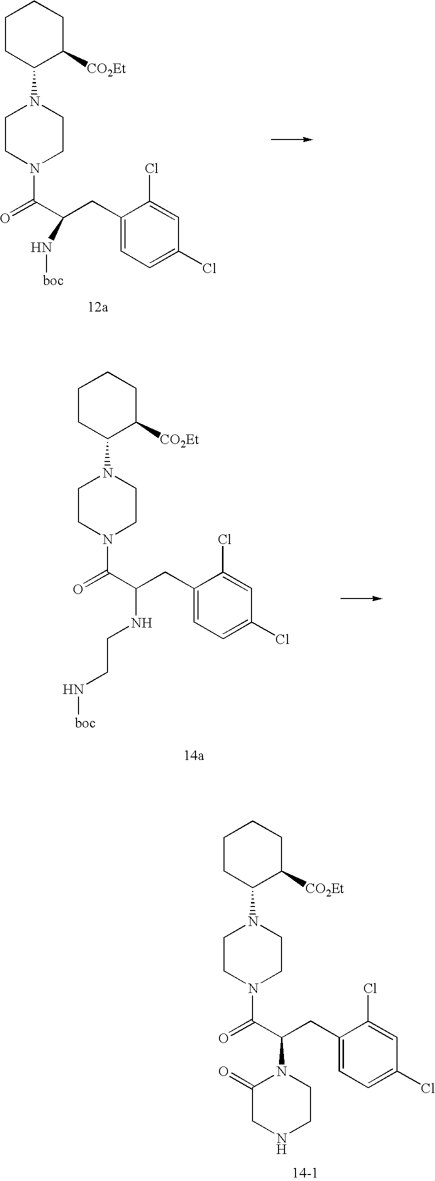

### Step 14A: trans-2-{4-[2-(N-Boc-amino)ethylamino-3-(4-chlorophenyl)propionyl]-piperazin-1-yl}-cyclohexanecarboxylic acid ethyl ester 14a

trans-2-{4-[2-(N-Boc-amino)-3-(4-chlorophenyl)propionyl]piperazin-1-yl}-cyclohexanecarboxylic acid ethyl ester 12a (2.2 mmol) was dissolved in dichloromethane (1 mL) and was treated with HCl (2.8 mL of a 4.0M solution in dioxane, 10.9 mmol). The resulting mixture was stirred at room temperature for 18 h then was concentrated under vacuum to give the crude amine hydrochloride salt as a yellow foam. This foam was dissolved in MeOH ( 11 mL) and dichloromethane ( 11 mL) and was treated with diisopropylethylamine (0.8 mL, 4.4 mmol). tert-Butyl N-(2-oxoethyl)carbamate (1.0 g, 6.3 mmol) was then added and the resulting mixture was stirred at room temperature for 1 h. NaBH₄ (0.25 g, 6.5 mmol) was then added portionwise over 15 minutes and the resulting mixture was stirred for 1 h. Another portion of tert-butyl N-(2-oxoethyl)carbamate (1.0g, 6.3mmol) was added, followed by more NaBH₄ (0.25 g, 6.5 mmol). The mixture was stirred at room temperature overnight and then worked-up. The crude residue was purified by column chromatography on silica gel, eluting with a 95:5 v/v mixture of EtOAc and MeOH. trans-2-{4-[2-(N-Boc-amino)ethylamino-3-(4-chlorophenyl)propionyl]piperazin-1-yl}-cyclohexanecarboxylic acid ethyl ester 14a was isolated as a white foam. LCMS m/z 498 (M⁺+1).

### Step 14B: trans-2-{4-[2-(2-Oxo-1-piperazinyl)-3-(4-chlorophenyl)propionyl]piperazin-1-yl}-cyclohexanecarboxylic acid ethyl ester 14-1

Chloroacetyl chloride (0.13 mL, 1.2 mmol) was added to a vigorously stirring suspension of trans-2-{4-[2-(N-Boc-amino)ethylamino-3-(4-chlorophenyl)propionyl]-piperazin-1-yl}-cyclohexanecarboxylic acid ethyl ester 14a (0.6 mmol) in EtOAc (4 mL) and aqueous saturated NaHCO₃ (4 mL). After 1.5 h, the organic layer was separated and concentrated under vaccum to give a white foam. This foam was treated with a 1:1 v/v solution of dichloromethane and trifluoroacetic acid for 1 hour at room temperature. The volatiles were removed under vacuum and the residue was dissolved in dichloromethane (50 mL) and washed with aqueous saturated NaHCO₃ and brine. The organic layer was dried over anhydrous MgSO₄, filtered and concentrated under vacuum. trans-2-{4-[2-(2-Oxo-1-piperazinyl)-3-(4-chlorophenyl)propionyl]piperazin-1-yl}-cyclohexanecarboxylic acid ethyl ester 14-1 was obtained as a yellow foam. LCMS m/z 539 (M⁺+1).

### Example 15

### N-ETHYL 4-[2-ACETAMIDO-3-(2,4-DICHLOROPHENYL)PROPIONYL]-1-PIPERAZINYLCYCLOHEXYLCARBOXYLIC AMIDE

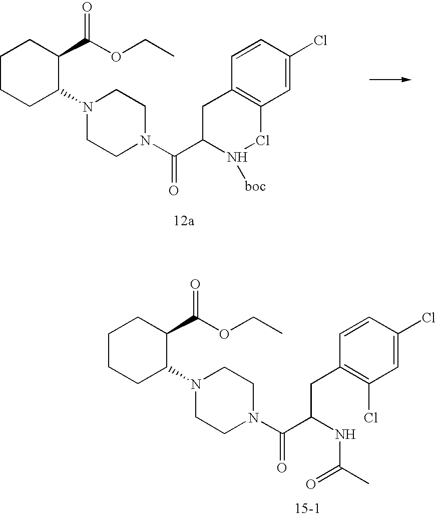

2-{4-[2-(N-Boc-amino)-3-(4-chlorophenyl)propionyl]piperazin-1-yl}-cyclohexanecarboxylic acid ethyl ester 12a was dissolved in dichloromethane (15 mL) along with 10 mL of 2M HCl in ether solution. The reaction mixture was stirred at room temperature for 4 hours then solvent was removed in vacuo. The deprotected amine was recovered as the HCl salt in 88% yield (0.97 g). This intermediate amine-HCl salt (2.1 mmol) was then dissolved in DMF (8 mL) along with acetic acid (2.1 mmol) and Et₃N (585 uL, 4.21 mmol), and treated with HBTU. The reaction mixture was stirred at room temperature for 2 hours then was washed with saturated NaHCO₃ solution (3×15 mL) and saturated NaCl solution (15 mL). The organic layer was separated, dried over anhydrous MgSO₄, filtered, and solvent was removed in vacuo. The residue was purified by column chromatography on silica using 50% ethyl acetate/hexanes as the eluent to give 15-1.

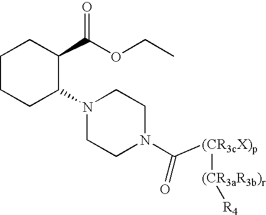

| Ex. # | —C(═O)—(CR_{3c}X)_{p—(CR}₃ₐR_{3b})ᵣ—R₄ | MS | MW |
|---|---|---|---|
| 15-1 | 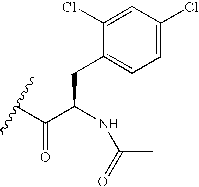 | 498.2 | 498.4 |
| 15-2 | 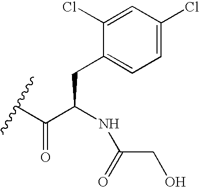 | 514.1 | 514.4 |
| 15-3 | 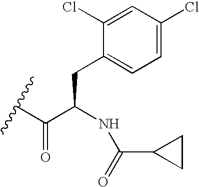 | 524.2 | 524.5 |
| 15-4 | 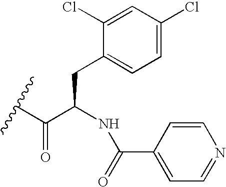 | 561.2 | 561.5 |
| 15-5 | 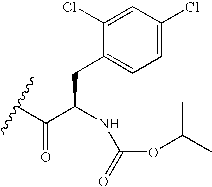 | 542.2 | 542.5 |
| 15-6 | 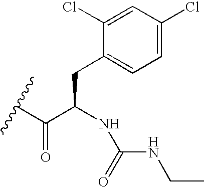 | 527.2 | 527.5 |
| 15-7 | 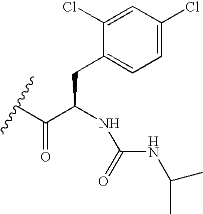 | 541.2 | 541.5 |
| 15-8 | 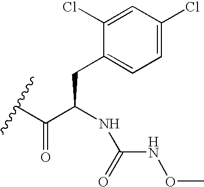 | 529.2 | 529.5 |

### Example 16

### N-ETHYL 4-[2-ACETAMIDO-3-(2,4-DICHLOROPHENYL)PROPIONYL]-1-PIPERAZINYLCYCLOHEXYLCARBOXYLIC AMIDE

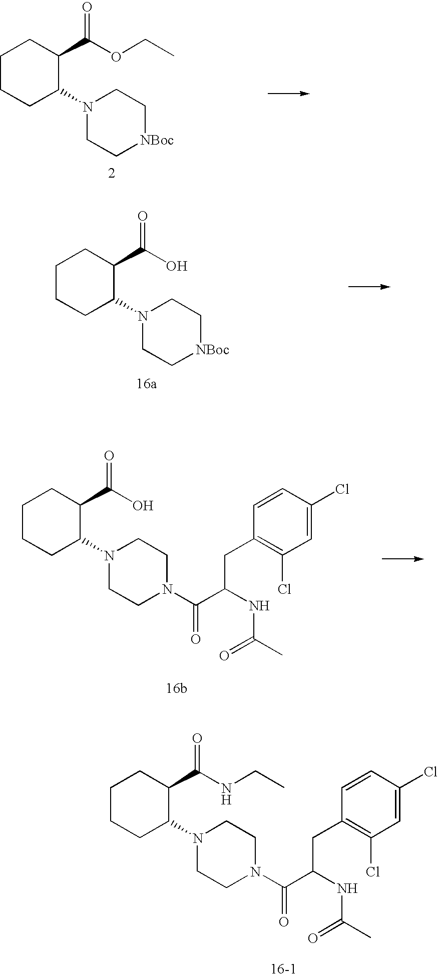

### Step 16A: 4-Boc-1-piperazinylcyclohexylcarboxylic acid 16a

4-Boc-1-piperazinylcyclohexylcarboxylic acid ethyl ester (3 g) was dissolved in a mixture of water (5 mL) and ethanol (5 mL) and was treated with KOH (1 g). The mixture was heated at reflux for 5 hours, cooled to room temperature and acidified with HCl to pH ˜5. The mix was extracted with ethyl acetate and the extract was washed with brine, dried and concentrated to give 4-Boc-1-piperazinylcyclohexylcarboxylic acid 16a.

### Step 16B: 4-[2-acetamido-3-(2,4-dichlorophenyl)propionyl]-1-piperazinylcyclohexyl-carboxylic acid 16b

4-Boc-1-piperazinylcyclohexylcarboxylic acid 16a was dissolved in dichloromethane (25 mL) and treated with 2M HCl/ether (40 mL). This mixture was stirred at room temperature for 4 hours, and then concentrated in vacuo. The residue was triturated with ether to precipitate 1-piperazinylcyclohexylcarboxylic acid as the HCl salt as a cream colored solid.

2-Acetamido-3-(2,4-dichlorophenyl)propionic acid (1.1 g) was dissolved in DMF (10 mL) and treated with HBTU (1.5 g) and DIEA (1.4 mL). This mixture was stirred at room temperature for 30 minutes. The above 1-piperazinylcyclohexylcarboxylic acid HCl salt (1 g) and DIEA (1.4 mL) were added and the reaction was stirred for an hour. The reaction was quenched with water and the product was extracted with ethyl acetate. The extract was washed with water (×2), brine, then dried over MgSO₄ and concentrated in vacuo to give 4-[2-acetamido-3-(2,4-dichlorophenyl)propionyl]-1-piperazinylcyclohexylcarboxylic acid 16b (2 g).

### Step 16C: N-Ethyl 4-[2-acetamido-3-(2,4-dichlorophenyl)propionyl]-1-piperazinyl-cyclohexylcarboxylic amide 16-1

A mixture of 4-[2-acetamido-3-(2,4-dichlorophenyl)propionyl]-1-piperazinylcyclohexylcarboxylic acid 16b (60 mg) and ethylamine (11 mg) in DMF (2 mL) was treated with HBTU (49 mg) and DIEA (46 μl) and the mixture was stirred at room temperature for two hours. N-Ethyl 4-[2-acetamido-3-(2,4-dichlorophenyl)propionyl]-1-piperazinylcyclohexylcarboxylic amide 16-1 was purified on HPLC. MS 497 (MH⁺).

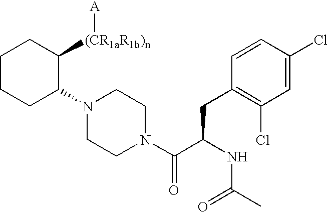

| Ex. # | —(CR₁ₐR_{1b})ₙ—A | MS | MW |
|---|---|---|---|
| 16-1 | 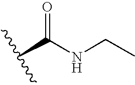 | 497.2 | 497.5 |
| 16-2 | 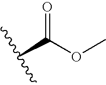 | 484.1 | 484.4 |
| 16-3 | 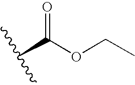 | 498.2 | 498.4 |
| 16-4 | 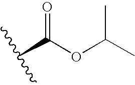 | 512.1 | 512.5 |
| 16-5 | 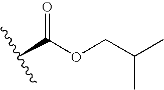 | 526.1 | 526.5 |
| 16-6 | 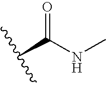 | 483.1 | 483.4 |
| 16-7 | 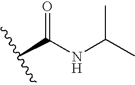 | 511.2 | 511.5 |
| 16-8 | 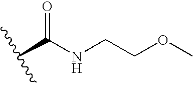 | 527.2 | 527.5 |
| 16-9 | 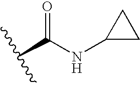 | 509.1 | 509.5 |
| 16-10 | 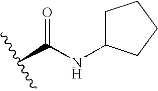 | 537.2 | 537.5 |
| 16-11 | 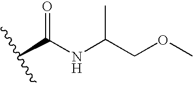 | 541.2 | 541.5 |
| 16-12 | 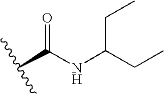 | 539.2 | 539.5 |
| 16-13 | 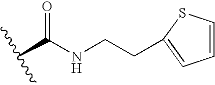 | 579.1 | 579.6 |
| 16-14 | 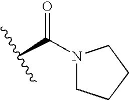 | 523.2 | 523.5 |
| 16-15 | 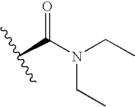 | 525.2 | 525.5 |
| 16-16 | 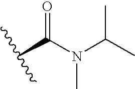 | 525.2 | 525.5 |
| 16-17 | 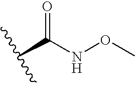 | 499.1 | 499.4 |

It will be appreciated that, although specific embodiments of the invention have been described herein for purposes of illustration, various modifications may be made without departing from the spirit and scope of the invention. Accordingly, the invention is not limited except as by the appended claims.

1. A compound having the following structure: 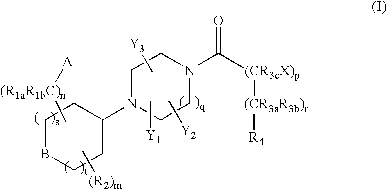
or a stereoisomer, prodrug or pharmaceutically acceptable salt thereof,
wherein:
A is —OR₅, —NR₆R₇, —C(═O)NR₆R₇, —C(═O)OR₈, —OC(═O)R₅, —OC(═O)NR₆R₇, —NR₆C(═O)OR₈, —NR₆C(═O)R₅, —NR₆C(═O)NR₆R₇, —NR₆SO₂R₉, —SO₂NR₆R₇, —NR₆SO₂NR₆R₇, —C(═NR₆)NR₆R₇, —C(O)NR₆C(═NR₆)NR₆R₇, —NR₆C(═NR₇)R₉, heterocycle or substitute heterocycle;
B is a direct bond, —O—, —S—, —S(═O—, or —S(═O)₂—;
m is 0, 1, or 2;
n is 0, 1, 2, or 3;
p is 0 or 1;
q is 1 or 2;
r is 0, 1, or 2;
s is 0, 1, or 2;
t is 0, 1, or 2;
X is, at each occurrence, independently hydrogen, hydroxy, fluorine, —OR₅, —NR₆R₇, —C(═O)NR₆R₇, —C(═O)OR₈, —OC(═O)R₅, —OC(═O)NR₆R₇, —NR₆C(═O)OR₈, —NR₆C(═O)R₅, —NR₆C(═O)NR₆R₇, —NR₆SO₂R₉, —SO₂NR₆R₇, —NR₆SO₂NR₆R₇, —C(═NR₆)NR₆R₇, —C(O)NR₆C(═NR₆)NR₆R₇, —NR₆C(═NR₇)R₉, heterocycle, or substituted heterocycle;
R₁ₐ and R_{1b} are, at each occurrence, the same or different and independently hydrogen, alkyl, substituted alkyl, aryl, substituted, aryl, hydroxy, amino, alkylamino, cyano, halide, —COOR₈, or —CONHR₆;
R₂ is, at each occurrence, independently alkyl, substituted alkyl, hydroxy, or halogen;
R₃ₐ, R_{3b}, and R_{3c} are, at each occurrence, the same or different and independently hydrogen, alkyl, or substituted alkyl;
R₄ is aryl, substituted aryl, heteroaryl, or substituted heteroaryl;
R₅ is, at each occurrence, independently hydrogen, hydroxy, alkyl, substituted alkyl, aryl, substituted aryl, heterocycle, or substituted heterocycle;
R₆ and R₇ are, at each occurrence, the same or different and independently hydrogen, alkyl, substituted alkyl, aryl, substituted aryl, arylalkyl, substituted arylalkyl, heterocycle, substituted heterocycle, heterocyclealkyl, or substituted heterocyclealkyl;
R₈ and R₉ are, at each occurrence, the same or different and independently hydrogen, alkyl, substituted alkyl, aryl, substituted aryl, arylalkyl, substituted arylalkyl, heterocycle, substituted heterocycle, heterocyclealkyl, or substituted heterocyclealkyl; and
Y₁, Y₂ and Y₃ are the same or different and independently hydrogen or alkyl, or Y₁ and Y₂ taken together are oxo.

2. The compound of claim 1 wherein B is a direct bond, O, or S.

3. The compound of claim 1 where B is a direct bond.

4. The compound of claim 2 wherein s is 1 and t is 2.

5. The compound of claim 2 wherein s is 2 and t is 2.

6. The compound of claim 1 wherein n is 1 or 2.

7. The compound of claim 6 wherein n is 1.

8. The compound of claim 1 wherein R₄ is substituted aryl.

9. The compound of claim 1 wherein each of Y₁, Y₂, and Y₃ are hydrogen.

10. The compound of claim 1 wherein A is —OR₅, —NR₆R₇, —C(═O)NR₆R₇, —C(═O)OR₈, —OC(═O)R₅, —OC(═O)NR₆R₇, —NR₆C(═O)OR₈, or —NR₆C(═O)R₅.

11. The compound of claim 10 where X is hydrogen, —NR₆R₇, —C(═O)NR₆R₇, —C(═O)OR₈, —OC(═O)R₅, —OC(═O)NR₆R₇, —NR₆C(═O)OR₈, —NR₆C(═O)R₅, —NR₆C(═O)NR₆R₇, —NR₆SO₂R₉, —SO₂NR₆R₇, or —NR₆SO₂NR₆R₇.

12. The compound of claim 1 wherein p is 1 and R_{3c} is hydrogen.

13. A pharmaceutical composition comprising a compound of claim 1 in combination with a pharmaceutically acceptable carrier.

14. A method for altering a disorder associated with the activity of a melanocortin receptor, comprising administering to a patient in need thereof an effective amount of a compound of claim 1.

15. The method of claim 14 wherein the melanocortin receptor is melanocortin 3 receptor.

16. The method of claim 14 where the melanocortin receptor is melanocortin 4 receptor.

17. The method of claim 14 wherein the compound is an antagonist of the melanocortin receptor.

18. The method of claim 14 wherein the compound is an agonist of the melanocortin receptor.

19. The method of claim 14 wherein the disorder is an eating disorder.

20. The method of claim 19 wherein the eating disorder is cachexia.

21. The method of claim 14 wherein the disorder is a sexual dysfunction.

22. The method of claim 21 where the sexual disfunction is erectile dysfunction.

23. The method of claim 14 wherein the disorder is a skin disorder.

24. The method of claim 14 where the disorder is chronic pain.

25. The method of claim 14 where the disorder is anxiety or depression.

26. The method of claim 14 wherein the disorder is obesity.
